(19) United States
(12) Patent Application Publication
(10) Pub. No.: US 2026/0146039 A1
(43) Pub. Date: May 28, 2026

(54) NEUROPLASTIGEN MODULATORS FOR THE TREATMENT OF PAIN, ITCH AND NERVE INJURY

(71) Applicant: Cacti, Inc., Flower Mound, TX (US)

(72) Inventors: David Roberson, Lewisville, TX (US); Kaitlin Roberson, Lewisville, TX (US); Robert B. Perni, Marlborough, MA (US)

(21) Appl. No.: 19/403,333

(22) Filed: Nov. 28, 2025

Related U.S. Application Data
(63) Continuation of application No. PCT/US24/31949, filed on May 31, 2024.
(60) Provisional application No. 63/470,061, filed on May 31, 2023.

(51) Int. Cl. C07D 471/06; A61K 31/48; A61P 25/00; C07D 471/20

(52) U.S. Cl. CPC C07D 471/06; A61K 31/48; A61P 25/00; C07D 471/20

(57) ABSTRACT

The present invention provides compounds represented by Formula (I),

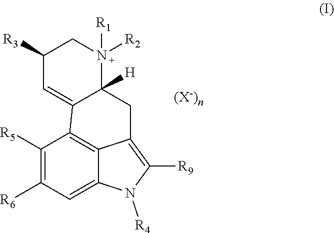

wherein R₁, R₂, R₃, R₄, R₅, R₆, R₉, X, and n are as defined herein. The invention further provides pharmaceutical compositions comprising such compounds and uses of such compounds for treating pain, itch or cough.

### RELATED APPLICATION

This application is a continuation of International Application No. PCT/US2024/031949, which designated the United States and was filed on May 31, 2024, published in English, which claims the benefit of U.S. Provisional Application No. 63/470,061 filed May 31, 2023. The entire contents of the above-referenced applications are incorporated by reference herein.

### BACKGROUND OF THE INVENTION

There are three general mechanisms responsible for the generation of pain. Nociceptive pain, which includes inflammatory pain, is generated by noxious stimuli that can result in tissue damage. The stimuli include thermal, stress/strain (impact), chemical and inflammatory agents. The second type of pain is neuropathic pain, which is caused by damage to peripheral somatosensory nerves. The broadness (heterogeneity) of that definition makes the treatment of neuropathic pain particularly challenging. The third type of pain is nociplastic pain which arises from altered nociception but without any identifiable present or potential tissue damage. All three categories of pain include conditions involving activation of nociceptors, which are peripheral sensory neurons that detect or contribute to the perception of pain and may express large-pore ion channels.

Neuropathic pain is quite common, impacting approximately 7% of individuals within the United States. It has no apparent protective biological role and is particularly challenging to treat. Neuropathic pain arises after peripheral nerve damage such as from trauma, surgical intervention, herniation of an intervertebral disc, spinal cord injuries, diabetes, herpes zoster (shingles) infection, HIV/AIDS, advanced-stage cancer, limb amputation (inclusive of mastectomy), carpal tunnel syndrome, habitual alcohol consumption, exposure to radiation, and as an unforeseen aftermath of neurotoxic therapeutic agents, such as specific anti-HIV and chemotherapeutic drugs. The sensory experience of neuropathic pain is often described as “burning”, “electric”, “tingling”, or “shooting” and can arise as allodynia—a condition where stimuli, such as a gentle touch, that typically do not provoke a painful response are painful—and hyperalgesia—an enhanced response to a stimulus that is usually painful, or spontaneous pain in the absence of any known stimulus, or that is perceived as much greater than would be expected from the known or observable injury or stimulus.

In all types of chronic pain, the transition from the acute to the chronic condition involves pain plasticity: maladaptive changes in the structure and function of pain circuits (Shraim M A, et al., Clin J Pain. 2020 October; 36 (10): 793-812). This plasticity can involve “central sensitization,” an umbrella term for facilitated synaptic plasticity in the central nervous system (i.e., brain, brainstem, and spinal cord) (Hoegh M., J Orthop Sports Phys Ther. 2023 January; 53 (1): 1-4) and peripheral sensitization which involves synaptic plasticity of peripheral nociceptor neurons. Other types of neuronal plasticity that underlie chronic pain include changes in the structure (morphology), gene expression and electrophysiology of nociceptors (Costigan M, et al., Annu Rev Neurosci. 2009; 32:1-32), phenotypic changes (non-nociceptors become pain-sensing) (Bráz J M, et al., Neuron. 2012 May 24; 74 (4): 663-75), increased expression of ion channels (Devor, M. Journal of Pain, 2006, 7 (1), S3-S12), and changes in the expression patterns of receptors and neurotransmitters (Peleshok J C, & Ribeiro-da-Silva A, Journal of Comparative Neurology, 2011, 519 (1), 49-63).

Serotonin (5HT) receptors expressed in central and peripheral pain circuits, including 5HT1, 5HT2, 5HT5, and 5HT6 subtypes play a role in the pathogenesis of neuropathic pain (Liu Q Q, et al., Pharmacol Res. 2020 September; 159:104949). For example, a disc puncture neuropathic pain model causes transient upregulation of genes that encode 5HT2A receptors during the immediate post-injury period (Fujioka Y, et al., J Orthop Surg (Hong Kong). 2016 April; 24 (1): 106-12). Similarly, in the formalin model of neuropathic pain, activation of 5HT2A receptors is associated with pain hypersensitivity and allodynia (Cervantes-Durán C, et al., Pharmacol Rep. 2016 April; 68 (2): 434-42). 5HT2A activation also plays a role in the mechanical hypersensitivity that occurs in the rat chronic constriction nerve injury model (Nitanda A, et al. Neurochem Int. 2005. 47 (6): 394-400), and in the rodent spared nerve injury model (De Gregorio D, et al., Pain. 2019 January; 160 (1): 136-150). 5HT2A receptors are also involved in nociceptive pain-related morphological reorganization of interneurons (Alba-Delgado C, et al., J Neurosci. 2018 Dec. 5; 38 (49): 10489-10504), and in plastic changes evoked by a persistent inflammatory pain model (Okamoto K, et al., Neuroscience. 2005; 130 (2): 465-74).

Conversely, antagonists of 5HT2A such as ketanserin have anti-nociceptive effects in the context of nerve injury (Wang D, et al., Eur J Pharmacol. 2010. 627 (1-3): 124-30). Together, these data suggest that 5HT2A activity is involved in the maladaptive plasticity that occurs after peripheral nerve injury, the symptomology of neuropathic pain, and that 5HT receptors could be a therapeutic target either by reducing the impact of maladaptive plasticity shortly after nerve injury or by way of therapeutically-induced plasticity to subsequently repair or reverse, in part or in whole, the maladaptive plastic changes that follow nerve injury.

It was recently shown that psychedelic molecules such as LSD, DMT and DOI induce neuronal plasticity via their activity on 5HT2A receptors, and that these changes are thought to underlie their antidepressant effects (Ly C, et al., Cell Rep. 2018 Jun. 12; 23 (11): 3170-3182). The activation of 5HT2A receptors by both psychedelic molecules including LSD and DMT as well as by non-psychedelic molecules such as serotonin can be explained by location bias, where the physical location of a receptor within a cell can bias its downstream activation properties. In the case of 5HT2A receptors, it was shown that intracellular 5HT2A receptors are responsible for their potentially therapeutic neuronal plasticity-inducing properties and that the lipophilicity of 5HT2A agonist molecules—that is, their ability to permeate the neuronal membrane to gain access to intracellular 5HT2A receptors—correlates with their neuroplastigenicity (Vargas M V, et al., Science. 2023 Feb. 17; 379 (6633): 700-706). Many other compounds introduce plasticity into pain circuits, including opioids, which sensitize pain circuitry and contribute to maladaptive plasticity. 5HT2A agonists, however, are different than opioids or other drugs of abuse known to produce maladaptive plasticity, in that the structural and functional plasticity produced by serotonergic psychedelics appears to be correlated to their beneficial therapeutic profiles (Ly C, et al., Cell Rep. 2018 Jun. 12; 23 (11): 3170-3182).

In addition to the therapeutic potential of psychedelics for mental health conditions including depression, PTSD, and anxiety (De Gregorio D, et al., J Neurosci. 2021 Feb. 3; 41 (5): 891-900), LSD and other 5HT2A agonists are also known to disrupt functional connections in brain regions associated with chronic pain, and their ability to facilitate beneficial reconnections may explain evidence of their benefit for chronic pain (Kooijman N I, et al., Pain Pract. 2023 April; 23 (4): 447-458), including neuropathic pain (Fadiman J, et al., J Psychoactive Drugs. 2019; 51 (2): 118-22). However, the finding that their neuroplastigenicity is correlated with their lipophilicity introduces challenges to the development of psychedelics devoid of psychoactive effects since lipophilic compounds are systemically distributed and easily cross the blood-brain barrier.

Pruritus, more commonly referred to as itch, is a dermatological phenomenon that can manifest either locally or generally, and often presents in association with skin lesions such as rashes, atopic eczema, and wheals. Itch is transmitted by small-diameter primary sensory neurons called pruriceptors that are morphologically and functionally similar to pain-sensing nociceptors in a number of respects, including but not limited to, the expression of TRPV1 and/or TRPA1 channels. The onset of itch is linked with a variety of circumstances and conditions including, but not restricted to, stress, anxiety, exposure to ultraviolet radiation from the sun, metabolic and endocrine disorders such as liver or kidney disease and hyperthyroidism, malignancies like lymphoma, reactions to drugs or food, parasitic and fungal infections, allergic responses, hematologic conditions such as polycythemia vera, dermatological ailments as well as ophthalmic conditions including dry eye syndrome and allergic conjunctivitis.

Cough is a defensive reflex designed to protect the airway from foreign bodies and to aid in the clearance of luminal debris. This reflex, however, can become maladaptive in a number of disease states leading to a non-productive dry cough where hyper- or allo-tussive states exist. Hyper- and allo-tussive states are often chronic in nature lasting greater than three months and can be manifested in many airway diseases states including asthma, COPD, asthma-COPD overlap syndrome (ACOS), interstitial pulmonary fibrosis (IPF) and lung cancer. In addition, inappropriate cough reflexes can be manifested acutely and chronically following viral infection. Furthermore, chronic cough can be idiopathic in nature with unknown etiology.

As with pain, itch, and cough (i.e., the behavioral response to itch of the airways) are signaled by activation of primary sensory neurons (pruriceptors) by a pruritogen (itch-generating compound) or as a result of damage to pruriceptors (neuropathic itch) (Steinhoff M, et al., Pain. 2019 May; 160 Suppl 1:S11-S16). Therefore, there are similarities in both the physiological role of small-diameter primary sensory neurons in pain and itch and in the therapeutic approaches that may prove beneficial for treating different types of pain and itch.

Pain-, itch- and cough-sensing neurons differ from other types of neurons in expressing (in most cases) the non-selective cation channels TRPV1, which is activated by painful heat or by capsaicin, the pungent ingredient in chili pepper, or TRPA1 which is activated by noxious cold and by allyl isothiocyanate (AITC). Other types of receptors selectively expressed in various types of pain-sensing (nociceptor) and itch-sensing (pruriceptor) neurons include but are not limited to TRPM8, and P2X(2/3) receptors. These presence of these large pore ion channels on nociceptors and pruriceptors, but not in other peripheral neurons, can be exploited to selectively deliver permanently charged sodium-channel blocking drugs to the intracellular space of these cells, in cells where these channels are active, or by directly activating the ion channels by a selective ligand (Binshtok A M, et al., Nature. 2007 Oct. 4; 449 (7162): 607-10), or by indirect opening of TRP channels secondary to activation of g-protein coupled receptors (GPCRs) expressed on or within the same neuron (Roberson D P, et al., Nat Neurosci. 2013 July; 16 (7): 910-8).

Despite the progress made in developing therapies aimed at pain and itch management, there remains a need for additional therapeutic agents, particularly those that can modify the course of the disease state or dysfunction underlying the itch or pain sensation.

### SUMMARY OF THE INVENTION

The present invention provides compounds represented by Formula (I),

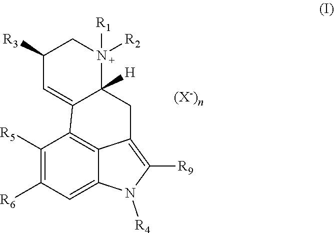

wherein
R₁ and R₂ are independently selected from the group consisting of substituted or unsubstituted C₁-C₁₂-alkyl, substituted or unsubstituted C₂-C₁₂-alkenyl; substituted or unsubstituted C₂-C₁₂-alkynyl; —CH₂CH₂—(OCH₂CH₂)ₘOR₁₂; and substituted or unsubstituted C₁-C₁₂-alkyl-Y; or one of R₁ and R₂ is —O⁻;

Alternatively, R₁ and R₂ are taken together with nitrogen atom to which they are attached to form a substituted or unsubstituted 3- to 8-membered heterocyclyl;
R₃ is selected from the group consisting of hydrogen, halogen, substituted or unsubstituted C₁-C₆-alkyl, —OR₈, —NR₇R₈, —C(O)NR₇R₈, —C(O)O⁻, and —C(O)OR₈;
R₄ is selected from the group consisting of hydrogen, substituted or unsubstituted C₁-C₆-alkyl, C₁-C₆-alkyl-OR₁₀, C₁-C₆-alkyl-NR₁₀R₁₁, —C(O)R₁₀, and —C(O)NR₁₀R₁₁;
R₅ and R₆ are independently selected from the group consisting of hydrogen, halogen, hydroxyl, substituted or unsubstituted C₁-C₆-alkyl, substituted or unsubstituted C₂-C₆-alkenyl; substituted or unsubstituted C₂-C₆-alkynyl, and C₁-C₆-alkoxy;
R₇ and R₈ are independently selected from the group consisting of hydrogen, substituted or unsubstituted C₁-C₈-alkyl; substituted or unsubstituted C₂-C₈-alkenyl; and substituted or unsubstituted C₂-C₈-alkynyl; or R₇ and R₈ are taken together with nitrogen atom to which they are attached to form a substituted or unsubstituted 3- to 8-membered heterocyclyl;
R₉ is selected from the group consisting of hydrogen, halogen, substituted or unsubstituted C₁-C₈-alkyl, substituted or unsubstituted C₂-C₈-alkenyl, and substituted or unsubstituted C₂-C₈-alkynyl;
R₁₀ and R₁₁ are independently selected from the group consisting of hydrogen, C₁-C₆-alkyl, substituted or unsubstituted C₂-C₆-alkenyl, and substituted or unsubstituted C₂-C₆-alkynyl;
R₁₂ is hydrogen or substituted or unsubstituted C₁-C₆-alkyl; preferably R₁₂ is hydrogen, methyl or —CF₃;
Y is selected from the group consisting of hydroxyl, substituted or unsubstituted C₁-C₆-alkoxy; substituted or unsubstituted C₁-C₆-alkyl-OC(O)—; and —C(O)O—; in certain embodiments, Y is —C(O)OH;
X⁻ is a pharmaceutically acceptable anion;
m is an integer from 1 to 5;
n is 0 when at least one of R₃ or Y is —C(O)O⁻; and
n is 1 when neither R₃ nor Y is —C(O)O.

The invention further provides pharmaceutical compositions comprising a compound of Formula (I) and methods of using a compound of Formula (I) for treating neuropathic pain, pruritis, or other forms of long-lasting pain that involve maladaptive plasticity.

### BRIEF DESCRIPTION OF THE DRAWINGS

FIG. 1A is a graph showing induction of calcium flux in cultured murine dorsal root ganglion (DRG) neurons by Compound 1 at 1 μM and 10 μM and controls.

FIG. 1B is a graph showing the percent of cultured murine dorsal root ganglion (DRG) neurons responsive to Compound 1 at 1 μM and 10 μM and controls.

FIG. 2A is a graph showing induction of calcium flux in cultured murine dorsal root ganglion (DRG) neurons by Compound 2 at 1 μM and 10 μM and controls.

FIG. 2B is a graph showing the percent of cultured murine dorsal root ganglion (DRG) neurons responsive to Compound 2 at 1 μM and 10 μM and controls.

FIG. 3A is a graph of average hind-left luminance in control, CFA plus Compound 1, and CFA plus PBS groups. * Indicates a significant difference from the CFA+PBS group (p<0.05). # Indicates a significant difference from the Control group (p<0.05).

FIG. 3B is a graph of hind-left usage in control, CFA plus Compound 1, and CFA plus PBS groups. * Indicates a significant difference from the CFA+PBS group (p<0.05). # Indicates a significant difference from the Control group (p<0.05).

FIG. 4A is a graph of average hind-right luminance in control, CFA plus Compound 1, and CFA plus PBS groups. * Indicates a significant difference from the CFA+PBS group (p<0.05). # Indicates a significant difference from the Control group (p<0.05).

FIG. 4B is a graph of hind-right usage in control, CFA plus Compound 1, and CFA plus PBS groups. * Indicates a significant difference from the CFA+PBS group (p<0.05). # Indicates a significant difference from the Control group (p<0.05).

FIG. 5A is a graph of average hind-left luminance in control, CFA plus Compound 2, and CFA plus PBS groups. * Indicates a significant difference from the CFA+PBS group (p<0.05). # Indicates a significant difference from the Control group (p<0.05).

FIG. 5B is a graph of hind-left usage in control, CFA plus Compound 2, and CFA plus PBS groups. * Indicates a significant difference from the CFA+PBS group (p<0.05). # Indicates a significant difference from the Control group (p<0.05).

FIG. 6A is a graph of average hind-right luminance in control, CFA plus Compound 2, and CFA plus PBS groups. * Indicates a significant difference from the CFA+PBS group (p<0.05). # Indicates a significant difference from the Control group (p<0.05).

FIG. 6B is a graph of hind-right usage in control, CFA plus Compound 2, and CFA plus PBS groups. * Indicates a significant difference from the CFA+PBS group (p<0.05). # Indicates a significant difference from the Control group (p<0.05).

### DETAILED DESCRIPTION OF THE INVENTION

The invention features novel ergoline alkaloid quaternary salts for inducing neuronal plasticity selectively in somatosensory neurons, immune cells, and other cell types by delivery of these charged, membrane-impermeable 5HT2A agonist compounds through a channel-forming receptor expressed on the extracellular surface of nociceptor and pruruiceptor neurons. In one embodiment, the present invention provides ergoline alkaloid quaternary salts which are nociplastic agents for the targeted treatment of non-nociceptive pain. These agents can be applied topically or parentally at the site of the pain. The compounds of the invention are cationic or zwitterionic and are designed to not penetrate the blood/brain barrier and consequently will produce no cognitive effects. In addition, the charge of these compounds is expected to preclude significant systemic exposure away from the pain site thereby increasing selectivity and safety. Moreover, the charge and hydrophobicity of these compounds are expected to prevent them from gaining entry to the intracellular space of most neurons except those peripheral nociceptors nearby the site of activation that express non-selective cation channel-forming receptors, such as TRPV1 and/or TRPA1. Thus, the compounds of the invention are not expected to substantially activate intracellular 5HT2A receptors in cells that do not express extracellular cation channels, except for those that express non-selective cation channel-forming receptors, such as TRPV1, TRPA1, P2X(2/3) or in cells that do not express extracellular cation channels, or in which the extracellular cation channels are not activated (not open).

In one embodiment, the present invention provides a compound of Formula (I) as described above.

In certain embodiments the compound of Formula (I) is not either compound set forth below.

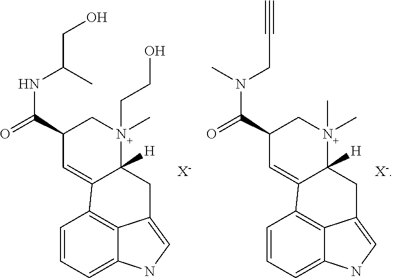

In certain embodiments, the compound of Formula (I) is represented by Formula (II),

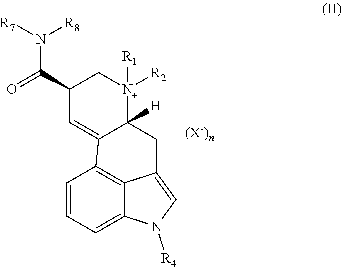

where R₁, R₂, R₄, R₇, R₈, X, and n have the meanings given above.

In certain embodiments of the compounds of Formula (I) and Formula (II), R₇ and R₈ are independently substituted or unsubstituted C₁-C₆-alkyl, preferably substituted or unsubstituted C₁-C₄-alkyl. In certain embodiments, R₇ and R₈ are both ethyl.

In certain embodiments of the compounds of Formula (I) and Formula (II), R₇ and R₈ are taken together with the nitrogen atom to which they are attached to form a substituted or unsubstituted saturated 3- to 8-membered heterocyclyl, preferably a substituted or unsubstituted saturated 4- to 6-membered heterocyclyl. In certain embodiments, R₇ and R₈ are taken together with the nitrogen atom to which they are attached to form a substituted or unsubstituted 1-piperidinyl, 1-pyrrolidinyl or 1-azetidinyl ring.

In certain embodiments of the compounds of Formula (I) and Formula (II), R₁ and R₂ are independently C₁-C₁₂-alkyl or C₁-C₁₂-alkyl-Y. In these embodiments, R₁ and R₂ are preferably independently C₁-C₆-alkyl or C₁-C₆-alkyl-Y. In certain embodiments of the compounds of Formula (I) and Formula (II), R₁ is C₁-C₁₂-alkyl and R₂ is C₁-C₁₂-alkyl-Y. In certain embodiments of the compounds of Formula (I) and Formula (II), R₁ is C₁-C₆-alkyl and R₂ is C₁-C₆-alkyl-Y. In these embodiments, Y is preferably hydroxyl, C₁-C₆-alkoxy, or —C(O)O⁻, more preferably hydroxyl; C₁-C₃-alkoxy, such as methoxy; or —C(O)O⁻. It is to be understood that when Y is represented as —C(O)O⁻, this group is the conjugate base of —C(O)OH, and can exist in either form, or a combination thereof, depending on the pH. When Y is —C(O)O⁻, the compound forms a zwitterion and X⁻ will be absent. In cases where Y is present as a —C(O)OH, X⁻ will be present, i.e., n is 1.

In certain embodiments of the compounds of Formula (I) and Formula (II), R₁ and R₂ are independently C₁-C₁₂-alkyl or —CH₂CH₂—(OCH₂CH₂)ₘOR₁₂, where m is as defined above. In certain embodiments, R₁ is C₁-C₁₂-alkyl and R₂ is —CH₂CH₂—(OCH₂CH₂)ₘOR₁₂. In certain embodiments, R₁ is C₁-C₆-alkyl and R₂ is —CH₂CH₂—(OCH₂CH₂)ₘOR₁₂. Preferably, R₁₂ is hydrogen or substituted or unsubstituted C₁-C₄-alkyl, more preferably hydrogen, methyl trifluoromethyl, or ethyl. In certain embodiments, m is 1 or 2.

In certain embodiments of the compounds of Formula (I) and Formula (II), R₁ and R₂ are taken together with the nitrogen atom to which they are attached to form a substituted or unsubstituted saturated 3- to 8-membered heterocyclyl, preferably a substituted or unsubstituted saturated 4- to 6-membered heterocyclyl. In certain embodiments, R₁ and R₂ are taken together with the nitrogen atom to which they are attached to form a substituted or unsubstituted piperidinyl, pyrrolidinyl or azetidinyl ring.

In certain embodiments of the compounds of Formula (I) and Formula (II), R₄ is selected from the group consisting of hydrogen and substituted or unsubstituted C₁-C₆-alkyl, such as substituted or unsubstituted methyl. Preferably, R₄ is hydrogen, methyl or trifluoromethyl. Most preferably, R₄ is hydrogen.

In certain embodiments of the compounds of Formula (I) and Formula (II), one of R₁ and R₂ is hydroxyl.

In certain embodiments, R₅ and R₆ are independently hydrogen, halogen, hydroxyl, substituted or unsubstituted C₁-C₄-alkyl and C₁-C₄-alkoxy. In certain embodiments, R₅ and R₆ are independently hydrogen, hydroxyl, halogen, methyl, trifluoromethyl, or methoxy. Preferably R₅ and R₆ are independently hydrogen, fluorine, or chlorine. More preferably, R₅ and R₆ are hydrogen.

In certain embodiments, R₉ is hydrogen, halogen, or substituted or unsubstituted C₁-C₄-alkyl. In certain embodiments, R₉ is hydrogen, fluorine, chlorine, methyl or trifluoromethyl.

The compounds of the invention include, but are not limited to, Compounds 1-16 set forth in the table below.

| | |
|---|---|
| 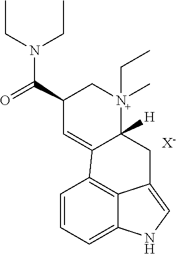 | 1 |
| 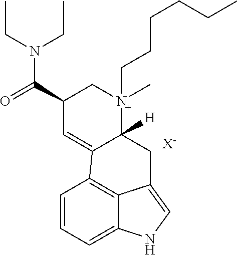 | 2 |
| 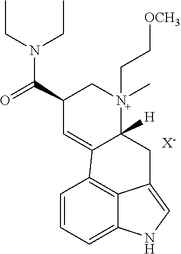 | 3 |
| 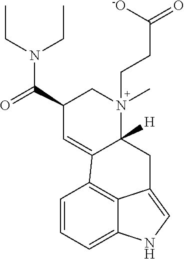 | 4 |
| 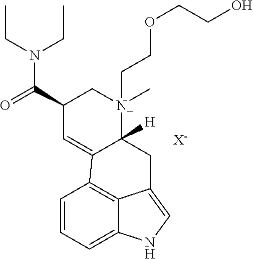 | 5 |
| 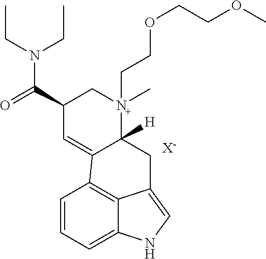 | 6 |
| 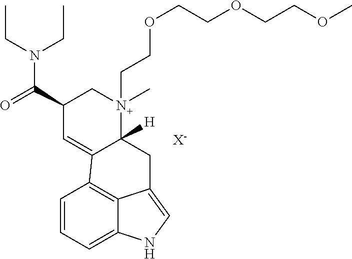 | 7 |
| 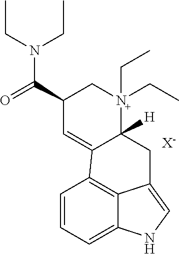 | 8 |
| 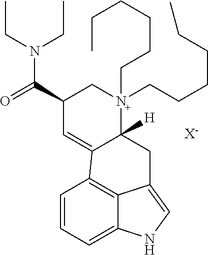 | 9 |
| 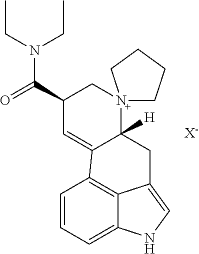 | 10 |
| 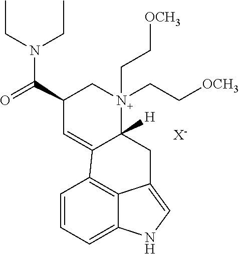 | 11 |
| 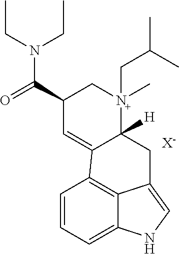 | 12 |
| 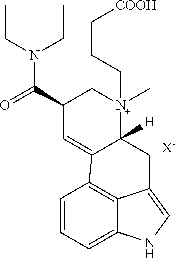 | 13 |
| 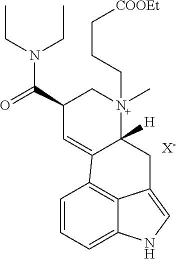 | 14 |
| 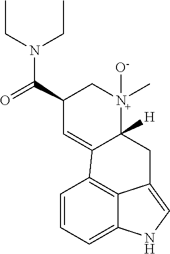 | 15 |
| 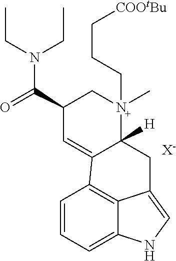 | 16 |

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be conducted in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. Tautomers may be cyclic or acyclic. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

Certain compounds of the present invention may also exist in different stable conformational forms which may be separable. Torsional asymmetry due to restricted rotation about an asymmetric single bond, for example because of steric hindrance or ring strain, may permit separation of different conformers. The present invention includes each conformational isomer of these compounds and mixtures thereof.

Additionally, the compounds of the present invention, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

Combinations of substituents and variables envisioned by this invention are only those that result in the formation of stable compounds. The term “stable,” as used herein, refers to compounds which possess stability sufficient to allow manufacture and which maintains the integrity of the compound for a sufficient period of time to be useful for the purposes detailed herein (e.g., therapeutic or prophylactic administration to a subject).

### Pharmaceutical Compositions

The invention further provides pharmaceutical compositions comprising a compound of Formula (I) or Formula (II) and a pharmaceutically acceptable carrier of excipient.

As used herein, the term “pharmaceutically acceptable carrier” means a non-toxic, inert solid, semi-solid or liquid filler, diluent, encapsulating material, or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and other animals orally, rectally, parenterally, intracisternally, intravaginally, intraperitoneally, topically (as by powders, ointments, or drops), buccally, or as an oral or nasal spray.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically acceptable carriers, adjuvants, or vehicles. In some cases, the pH of the formulation may be adjusted with pharmaceutically acceptable acids, bases, or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups, and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

To prolong the effect of a drug, it is often desirable to slow the absorption of the drug from subcutaneous or intramuscular injection. This may be accomplished using a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a compound of this invention include ointments, pastes, creams, lotions, gels, powders, solutions, sprays, inhalants, or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams, and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Methods of Treating Pain, Itch, and Cough

The invention further provides methods for treating pain, cough, itch, or a nerve injury in a subject in need thereof, comprising administering to the subject a therapeutically effective amount of a compound of the invention.

In certain embodiments, the method of the invention is for treating pain, such as pain selected from the group consisting of neuropathic pain, inflammatory pain, nociceptive pain, pain due to infections, and procedural pain, or wherein the neurogenic inflammatory disorder is selected from the group consisting of allergic inflammation, asthma, chronic cough, conjunctivitis, rhinitis, psoriasis, inflammatory bowel disease, interstitial cystitis, and atopic dermatitis.

Without being bound by theory, it is believed that the compounds of the invention are capable of passing through open large pore channels that are expressed on nociceptors and/or cough receptors and/or pruriceptors but not on motor neurons. Because the compounds of the invention are positively charged, they are not membrane-permeable and thus cannot enter cells that do not express large pore channels. Since large pore channels are often more active in tissue conditions associated with pain (such as inflammation) due to release of endogenous ligands, or activation by thermal stimuli, or plastic changes to the morphology or physiology of the nociceptors, the compounds of the invention can be used alone to selectively target activated nociceptors in order to effectively induce reparative or beneficial neuronal plasticity for the treatment (e.g., elimination or alleviation) of pain, cough, itch, or nerve damage. The compounds of the invention can also be used in combination with one or more exogenous large pore receptor agonists to selectively target nociceptors in order to effectively treat (e.g., eliminate or alleviate) pain, cough, itch, or neurogenic inflammation.

Under certain circumstances, large pore channels are activated without exposure to exogenous large pore channel agonists/ligands by endogenous inflammatory activators that are generated by tissue damage, infection, autoimmunity, atopy, ischemia, hypoxia, cellular stress, immune cell activation, immune mediator production, and oxidative stress, or by aberrant neuronal plasticity that changes the likelihood of spontaneous activation of these channels. Under such conditions, endogenous molecules (e.g., protons, lipids, and reactive oxygen species) can activate large pore channels expressed on nociceptors, allowing membrane-impermeable, 5HT agonists to gain access to the inside of the nociceptor through the endogenously-activated large pore channels. Endogenous inflammatory activators of large pore channels also include prostaglandins, nitric oxide (NO), peroxide (H2O2), cysteine-reactive inflammatory mediators like 4-hydroxynonenal, protons, ATP, endogenous alkenyl aldehydes, endocannabinoids, and immune mediators (e.g., interleukin 1 (IL-1), nerve growth factor (NGF), and bradykinin, whose receptors are coupled to large pore channels.

In certain embodiments, an exogenous agent which opens the large pore channel is co-administered with the compound of the invention. Thus, in certain embodiments the method of the invention further comprises administering to the subject an exogenous agent that permits passage of the 5HT agonist through the large pore ion channel (e.g., TRP channel) into the cell. Suitable such agents include capsaicin, another capsaicinoid, allyl isothiocyanate (AITC), or lidocaine. TRP channels may also be active in response to exogenous irritant activators such as inhaled acrolein from smoke or chemical warfare agents such as tear gas. The compound of the invention and the exogenous agent are preferably administered in amounts which in combination are therapeutically effective.

In certain embodiments, the present invention provides a method of activating 5HT2A receptors in a subject in need thereof, comprising administering to the subject an effective amount of a compound of Formula (I) or Formula (II). Preferably, the 5HT2A receptors to be activated are intracellular. In addition, it is preferred that 5HT2A receptors are activated locally without substantial systemic activation of 5HT2A receptors or such receptors in the central nervous system. This method is useful for treatment of a disease or disorder for which activation of 5HT2A receptors provides a therapeutic or palliative effect. In certain embodiments, the compound of Formula (I) or Formula (II) is administered topically.

In certain embodiments, the present invention provides a method of treating neuropathic pain or pruritis in a subject in need thereof. The method comprises the step of administering to the subject a therapeutically effective amount of a compound of Formula (I) or Formula (II).

In certain embodiments, the method is for treating neuropathic pain. The neuropathic pain can be associated with, for example, diabetic peripheral neuropathy, chemotherapy-induced peripheral neuropathy, nerve injury, and erythromelalgia. In certain embodiments, the neuropathic pain is localized to one or more anatomic sites. In these embodiments, the compound of Formula (I) or Formula (II) is preferably administered topically, preferably proximal to the site of pain.

In certain embodiments, the method is for treating pruritis. In certain embodiments, the pruritis is pruriplastic itch.

In certain embodiments, the method is for treating pain associated to a neuropathy or other nerve injury where the therapeutically effective amount of a compound of the invention is preferably delivered via topical or cutaneous application or through administration to conjunctiva or membranous epithelia. Such pain includes but is not limited to neuropathic pain secondary to: metabolic disorders such as peripheral diabetic neuropathy; neuropathy secondary to viral infections, including postherpetic neuralgia, HIV, and leprosy; neuropathic pain secondary to autoimmune disorders, for example multiple sclerosis, Guillain-Barre syndrome, chemotherapy-induced peripheral neuropathies, inflammatory disorder, hereditary neuropathies, and channelopathies; painful traumatic mononeuropathy; neuropathic pain secondary to facial nerve problems; shingles; and neuropathic pain secondary to nerve damage related to cosmetic surgical procedures including dermatological procedures, biopsies, and setting fractures, implants, or scoliosis rods.

In certain embodiments, the method is for treating pain or itch localized to one or more anatomic sites wherein the therapeutically effective amount of the compound of the invention is preferably delivered through cutaneous application or by administration to membranous epithelia including the conjunctiva. Such pain or itch includes or is associated with vulvodynia; endometriosis; chronic abdominal wall pain; complex regional pain syndrome; chronic pelvic inflammatory syndrome; testicular pain (orchialgia); perianal pain; hemorrhoid pain; urethritis; urological pain; lasting pain in scar tissue; itching from scar tissue; ocular pain; ocular itch; dry eye syndrome; eye redness; ophthalmic irritation; conjunctivitis; burn pain; pruritus (including, but not limited to, brachioradial, chronic idiopathic, genital/anal, notalgia paresthetica, and scalp); allergic dermatitis; atopic dermatitis; contact dermatitis; poison ivy; itch due to infections, parasites, insect bites, pregnancy, metabolic disorders, liver or renal failure, drug reactions, allergic reactions, eczema, hand eczema genital or anal itch, hemorrhoid itch, and cancer.

In certain embodiments, the method is for treating localized or regional pain where the therapeutically effective amount of a compound of the invention is preferably delivered via injection into the tissue or cavity where the pain is perceived or at a site proximal to the perceived pain, such as perineural, intra-articular, intramuscular, or subcutaneous injection. Such pain includes or is associated with: back pain; neck pain; lower back pain; cancer pain; gynecological and labor pain; tendonitis; achilles tendonitis; joint pain such as wrist, knee, hip, shoulder, or spine pain; temporomandibular disorders; interstitial cystitis; abdominal wall pain; chronic abdominal wall pain; colitis; gastritis; pancreatitis; pelvic floor pain (pea sized knots in the pelvic floor and muscle tightness that persists causing painful intercourse); dental pain; orthopedic pain; osteoarthritis; rheumatoid arthritis; back pain; neck pain; spinal compression; fracture or inflammation; nerve compression or infiltration by tumors; herniated disc; bulging disc; ruptured disc; compression of the spinal cord; compression of the sciatic nerve; trigeminal nerve damage; trigeminal trophic syndrome; cancer pain near a tumor; nerve compression or infiltration by tumors; muscle pain such as fibromyalgia; root avulsions; headaches, including migraines; painful polyneuropathy (including neuropathy due to diabetes or chemotherapy); central pain syndromes (potentially caused by virtually any lesion at any level of the nervous system); postsurgical pain syndromes (e.g., postmastectomy syndrome, post thoracotomy syndrome, phantom limb pain); visceral pain; nerve compression or infiltration by tumors; trauma or surgeries with resulting nerve damage; amputation with resulting numbness or tingling or nerve damage; stomach pain; stomach ulcers; burn pain or itch; postsurgical or post-procedural pain (e.g., postmastectomy syndrome, post thoracotomy syndrome, phantom limb pain); radicular pain; trauma or surgeries with resulting nerve damage; arthroscopy pain; neuropathic pain secondary to metabolic disorders such as peripheral diabetic neuropathy; neuropathy secondary to viral infections, including postherpetic neuralgia, HIV, and leprosy; neuropathic pain secondary to autoimmune disorders, for example multiple sclerosis, Guillain-Barre syndrome, chemotherapy-induced peripheral neuropathies, inflammatory disorder, hereditary neuropathies, channelopathies; painful traumatic mononeuropathy; facial nerve problems; shingles; nerve damage related to cosmetic surgical procedures including dermatological procedures, biopsies, and setting fractures, implants, scoliosis rods.

In certain embodiments, the method is for treating pain and painful disorders of the alimentary tract where the therapeutically effective amount of the compound of the invention is preferably delivered orally or via suppository to the gut epithelia. Such pain includes, or is associated with oral mucositis, esophagitis, gastritis, inflammatory bowel diseases, irritable bowel syndrome, Crohn's disease, dental pain, stomach pain, and stomach ulcers.

In certain embodiments, the method is for treating cough, airway inflammation, or other airway discomfort, wherein the therapeutically effective amount of the compound of the invention is preferably delivered to the airways, for example, in the form of a nebulizer, a dry powder inhaler or other inhaled formulation. Such cough, airway inflammation, or other airway discomfort includes, or is associated with asthma, COPD, asthma-COPD overlap syndrome (ACOS), interstitial pulmonary fibrosis (IPF), idiopathic cough, and lung cancer.

### Definitions

Listed below are definitions of various terms used to describe this invention. These definitions apply to the terms as they are used throughout this specification and claims, unless otherwise limited in specific instances, either individually or as part of a larger group.

The term “aryl,” as used herein, refers to a mono-, bi-, or polycyclic carbocyclic ring system comprising at least one aromatic ring, including, but not limited to, phenyl, naphthyl, tetrahydronaphthyl, indanyl, and indenyl. A polycyclic aryl is a polycyclic ring system that comprises at least one aromatic ring. Polycyclic aryls can comprise fused rings, covalently attached rings or a combination thereof.

The term “heteroaryl,” as used herein, refers to a mono-, bi-, or polycyclic aromatic radical having one or more ring atom selected from S, O and N; and the remaining ring atoms are carbon, wherein any N or S contained within the ring may be optionally oxidized. Heteroaryl includes, but is not limited to, pyridinyl, pyrazinyl, pyrimidinyl, pyrrolyl, pyrazolyl, imidazolyl, thiazolyl, oxazolyl, isoxazolyl, thiadiazolyl, oxadiazolyl, thiophenyl, furanyl, quinolinyl, isoquinolinyl, benzimidazolyl, benzoxazolyl, quinoxalinyl. A polycyclic heteroaryl can comprise fused rings, covalently attached rings or a combination thereof.

In accordance with the invention, aromatic groups can be substituted or unsubstituted.

The term “bicyclic aryl” or “bicyclic heteroaryl” refers to a ring system consisting of two rings wherein at least one ring is aromatic; and the two rings can be fused or covalently attached.

The term “alkyl” as used herein, refers to saturated, straight- or branched-chain hydrocarbon radicals. “C₁-C₃ alkyl”, “C₁-C₆ alkyl”, “C₁-C₁₀ alkyl”, “C₂-C₄ alkyl,” and “C₃-C₆ alkyl,” refer to alkyl groups containing from one to three, one to six, one to ten carbon atoms, 2 to 4 and 3 to 6 carbon atoms, respectively. Examples of C₁-C₈ alkyl radicals include, but are not limited to, methyl, ethyl, propyl, isopropyl, n-butyl, ter-butyl, neopentyl, n-hexyl, heptyl and octyl radicals.

The term “alkenyl” as used herein, refers to straight- or branched-chain hydrocarbon radicals having at least one carbon-carbon double bond by the removal of a single hydrogen atom. “C₂-C₁₀ alkenyl,” “C₂-C₈ alkenyl,” “C₂-C₄ alkenyl,” or “C₃-C₆ alkenyl”, refer to alkenyl groups containing from two to ten, two to eight, two to four or three to six carbon atoms, respectively. Alkenyl groups include, but are not limited to, for example, ethenyl, propenyl, butenyl, 1-methyl-2-buten-1-yl, heptenyl, octenyl, and the like.

The term “alkynyl” as used herein, refers to straight- or branched-chain hydrocarbon radicals having at least one carbon-carbon triple bond by the removal of a single hydrogen atom. “C₂-C₁₀ alkynyl,” “C₂-C₈ alkynyl,” “C₂-C₄ alkynyl,” or “C₃-C₆ alkynyl,” refer to alkynyl groups containing from two to ten, two to eight, two to four or three to six carbon atoms, respectively. Representative alkynyl groups include, but are not limited to, for example, ethynyl, 1-propynyl, 1-butynyl, heptynyl, octynyl, and the like.

The term “cycloalkyl,” as used herein, refers to a monocyclic or polycyclic saturated carbocyclic ring or a bi- or tri-cyclic group fused, bridged or spiro system, and the carbon atoms may be optionally oxo-substituted or optionally substituted with exocyclic olefinic, iminic or oximic double bond. Preferred cycloalkyl groups include C₃-C₁₂ cycloalkyl, C₃-C₆ cycloalkyl, C₃-C₈ cycloalkyl and C₄-C₇ cycloalkyl. Examples of C₃-C₁₂ cycloalkyl include, but not limited to, cyclopropyl, cyclobutyl, cyclopentyl, cyclohexyl, cyclopentyl, cyclooctyl, 4-methylene-cyclohexyl, bicyclo[2.2.1]heptyl, bicyclo[3.1.0]hexyl, spiro[2.5]octyl, 3-methylenebicyclo[3.2.1]octyl, spiro[4.4]nonanyl, and the like.

The term “cycloalkenyl,” as used herein, refers to monocyclic or polycyclic carbocyclic ring or a bi- or tri-cyclic group fused, bridged or spiro system having at least one carbon-carbon double bond and the carbon atoms may be optionally oxo-substituted or optionally substituted with exocyclic olefinic, iminic or oximic double bond. Preferred cycloalkenyl groups include C₃-C₁₂ cycloalkenyl, C₃-C₈ cycloalkenyl or C₅-C₇cycloalkenyl groups. Examples of C₃-C₁₂ cycloalkenyl include, but not limited to, cyclopropenyl, cyclobutenyl, cyclopentenyl, cyclohexenyl, cycloheptenyl, cyclooctenyl, bicyclo[2.2.1]hept-2-enyl, bicyclo[3.1.0]hex-2-enyl, spiro[2.5]oct-4-enyl, spiro[4.4]non-1-enyl, bicyclo[4.2.1]non-3-en-9-yl, and the like.

As used herein, the term “arylalkyl” means a functional group wherein an alkylene chain is attached to an aryl group, e.g., —CH₂CH₂-phenyl. The term “substituted arylalkyl” means an arylalkyl functional group in which the aryl group is substituted. Similarly, the term “heteroarylalkyl” means a functional group wherein an alkylene chain is attached to a heteroaryl group. The term “substituted heteroarylalkyl” means a heteroarylalkyl functional group in which the heteroaryl group is substituted.

As used herein, the term “alkoxy” employed alone or in combination with other terms means, unless otherwise stated, an alkyl group having the designated number of carbon atoms connected to the rest of the molecule via an oxygen atom, such as, for example, methoxy, ethoxy, 1-propoxy, 2-propoxy (isopropoxy) and the higher homologs and isomers. Preferred alkoxy are (C₁-C₃) alkoxy.

It is understood that any alkyl, alkenyl, alkynyl, cycloalkyl, heterocyclyl and cycloalkenyl moiety described herein can also be an aliphatic group or an alicyclic group.

An “aliphatic” group is a non-aromatic moiety comprised of any combination of carbon atoms, hydrogen atoms, halogen atoms, oxygen, nitrogen, or other atoms, and optionally contains one or more units of unsaturation, e.g., double and/or triple bonds. Examples of aliphatic groups are functional groups, such as alkyl, alkenyl, alkynyl, O, OH, NH, NH₂, C(O), S(O)₂, C(O)O, C(O)NH, OC(O)O, OC(O)NH, OC(O)NH₂, S(O)₂NH, S(O)₂NH₂, NHC(O)NH₂, NHC(O)C(O)NH, NHS(O)₂NH, NHS(O)₂NH₂, C(O)NHS(O)₂. C(O)NHS(O)₂NH or C(O)NHS(O)₂NH₂, and the like, groups comprising one or more functional groups, non-aromatic hydrocarbons (optionally substituted), and groups wherein one or more carbons of a non-aromatic hydrocarbon (optionally substituted) is replaced by a functional group. Carbon atoms of an aliphatic group can be optionally oxo-substituted. An aliphatic group may be straight chained, branched, cyclic, or a combination thereof and preferably contains between about 1 and about 24 carbon atoms, more typically between about 1 and about 12 carbon atoms. In addition to aliphatic hydrocarbon groups, as used herein, aliphatic groups expressly include, for example, alkoxyalkyls, polyalkoxyalkyls, such as polyalkylene glycols, polyamines, and polyimines, for example. Aliphatic groups may be optionally substituted.

The term “carbocycle” or “carbocyclic” refers to a saturated, partially unsaturated, or aromatic cyclic group in which each atom within the ring is carbon. Examples of carbocyclics include cycloalkyl, cycloalkenyl and aryl groups.

The terms “heterocyclyl” or “heterocycloalkyl” can be used interchangeably and referred to a non-aromatic ring or a bi- or tri-cyclic group fused, bridged or spiro system, where (i) each ring system contains at least one heteroatom independently selected from oxygen, sulfur and nitrogen, (ii) each ring system can be saturated or unsaturated (iii) the nitrogen and sulfur heteroatoms may optionally be oxidized, (iv) the nitrogen heteroatom may optionally be quaternized, (v) any of the above rings may be fused to an aromatic ring, and (vi) the remaining ring atoms are carbon atoms which may be optionally oxo-substituted or optionally substituted with exocyclic olefinic, iminic or oximic double bond. Representative heterocycloalkyl groups include, but are not limited to, 1,3-dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, 2-azabicyclo[2.2.1]-heptyl, 8-azabicyclo[3.2.1]octyl, 5-azaspiro[2.5]octyl, 1-oxa-7-azaspiro[4.4]nonanyl, 7-oxooxepan-4-yl, and tetrahydrofuryl. Such heterocyclyl groups may be further substituted. Heteroaryl or heterocyclyl groups can be C-attached or N-attached (where possible).

It is understood that any alkyl, alkenyl, alkynyl, alicyclic, cycloalkyl, cycloalkenyl, aryl, heteroaryl, heterocyclyl, aliphatic moiety or the like, described herein can also be a divalent or multivalent group when used as a linkage to connect two or more groups or substituents, which can be at the same or different atom(s). One skilled in the art can readily determine the valence of any such group from the context in which it occurs.

The term “substituted” refers to substitution by independent replacement of one, two, or three or more of the hydrogen atoms with substituents including, but not limited to, —F, —Cl, —Br, —I, —OH, —C₁-C₁₂-alkyl; —C₂-C₁₂-alkenyl, —C₂-C₁₂-alkynyl, —C₃-C₁₂-cycloalkyl, protected hydroxy, —NO₂, —N₃, —CN, —NH₂, protected amino, oxo, thioxo, —NH—C₁-C₁₂-alkyl, —NH—C₂-C₈-alkenyl, —NH—C₂-C₈-alkynyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₈-alkenyl, —O—C₂-C₈-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₈-alkenyl, —C(O)—C₂-C₈-alkynyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₈-alkenyl, —CONH—C₂-C₈-alkynyl, —CONH—C₃-C₁₂-cycloalkyl, —CONH-aryl, —CONH-heteroaryl, —CONH-heterocycloalkyl, —OCO₂—C₁-C₁₂-alkyl, —OCO₂—C₂-C₈-alkenyl, —OCO₂—C₂-C₈-alkynyl, —OCO₂—C₃-C₁₂-cycloalkyl, —OCO₂-aryl, —OCO₂-heteroaryl, —OCO₂-heterocycloalkyl, —CO₂—C₁-C₁₂ alkyl, —CO₂—C₂-C₈ alkenyl, —CO₂—C₂-C₈ alkynyl, CO₂—C₃-C₁₂-cycloalkyl, —CO₂-aryl, CO₂-heteroaryl, CO₂-heterocyloalkyl, —OCONH₂, —OCONH—C₁-C₁₂-alkyl, —OCONH—C₂-C₈-alkenyl, —OCONH—C₂-C₈-alkynyl, —OCONH—C₃-C₁₂-cycloalkyl, —OCONH-aryl, —OCONH-heteroaryl, —OCONH-heterocyclo-alkyl, —NHC(O)H, —NHC(O)—C₁-C₁₂-alkyl, —NHC(O)—C₂-C₈-alkenyl, —NHC(O)—C₂-C₈-alkynyl, —NHC(O)—C₃-C₁₂-cycloalkyl, —NHC(O)-aryl, —NHC(O)-heteroaryl, —NHC(O)-heterocyclo-alkyl, —NHCO₂—C₁-C₁₂-alkyl, —NHCO₂—C₂-C₈-alkenyl, —NHCO₂—C₂-C₈-alkynyl, —NHCO₂—C₃-C₁₂-cycloalkyl, —NHCO₂-aryl, —NHCO₂-heteroaryl, —NHCO₂-heterocycloalkyl, —NHC(O)NH₂, —NHC(O)NH—C₁-C₁₂-alkyl, —NHC(O)NH—C₂-C₈-alkenyl, —NHC(O)NH—C₂-C₈-alkynyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, —NHC(S)NH—C₁-C₁₂-alkyl, —NHC(S)NH—C₂-C₈-alkenyl, —NHC(S)NH—C₂-C₈-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₈-alkenyl, —NHC(NH)NH—C₂-C₈-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, —NHC(NH)—C₁-C₁₂-alkyl, —NHC(NH)—C₂-C₈-alkenyl, —NHC(NH)—C₂-C₈-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₈-alkenyl, —C(NH)NH—C₂-C₈-alkynyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₈-alkenyl, —S(O)—C₂-C₈-alkynyl, —S(O)—C₃-C₁₂-cycloalkyl, —S(O)-aryl, —S(O)-heteroaryl, —S(O)-heterocycloalkyl, —SO₂NH₂, —SO₂NH—C₁-C₁₂-alkyl, —SO₂NH—C₂-C₈-alkenyl, —SO₂NH—C₂-C₈-alkynyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₈-alkenyl, —NHSO₂—C₂-C₈-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₈-alkenyl, —S—C₂-C₈-alkynyl, —S—C₃-C₁₂-cycloalkyl, —S-aryl, —S-heteroaryl, —S-heterocycloalkyl, or methylthio-methyl. In certain embodiments, the substituents are independently selected from halo, preferably Cl and F; C₁-C₄-alkyl, preferably methyl and ethyl; halo-C₁-C₄-alkyl, such as fluoromethyl, difluoromethyl, and trifluoromethyl; C₂-C₄-alkenyl; halo-C₂-C₄-alkenyl; C₃-C₆-cycloalkyl, such as cyclopropyl; C₁-C₄-alkoxy, such as methoxy and ethoxy; halo-C₁-C₄-alkoxy, such as fluoromethoxy, difluoromethoxy, and trifluoromethoxy, —CN; —OH; NH₂; C₁-C₄-alkylamino; di(C₁-C₄-alkyl)amino; and NO₂. It is understood that the aryls, heteroaryls, alkyls, and the like can be further substituted. In some cases, each substituent in a substituted moiety is additionally optionally substituted when possible with one or more groups, each group being independently selected from C₁-C₄-alkyl; —CF₃, —OCH₃, —OCF₃, —F, —Cl, —Br, —I, —OH, —NO₂, —CN, and —NH₂.

In certain embodiments, a substituted alkyl, alkenyl or alkoxy group is substituted with one or more halogen atoms, preferably fluorine or chlorine atoms. Such substituted alkyl groups include fluoromethyl, difluoromethyl and trifluoromethyl. Such substituted alkoxy groups include fluoromethoxy, difluoromethoxy and trifluoromethoxy.

The term “halo” or halogen” alone or as part of another substituent, as used herein, refers to a fluorine, chlorine, bromine, or iodine atom.

The term “optionally substituted,” as used herein, means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In certain embodiments, the compounds of each formula herein are defined to include isotopically labelled compounds. An “isotopically labelled compound” is a compound in which at least one atomic position is enriched in a specific isotope of the designated element to a level which is significantly greater than the natural abundance of that isotope. For example, one or more hydrogen atom positions in a compound can be enriched with deuterium to a level which is significantly greater than the natural abundance of deuterium, for example, enrichment to a level of at least 1%, preferably at least 20% or at least 50%. Such a deuterated compound may, for example, be metabolized more slowly than its non-deuterated analog, and therefore exhibit a longer half-life when administered to a subject. Such compounds can synthesize using methods known in the art, for example by employing deuterated starting materials. Unless stated to the contrary, isotopically labelled compounds are pharmaceutically acceptable.

The term “hydroxy activating group,” as used herein, refers to a labile chemical moiety which is known in the art to activate a hydroxyl group so that it will depart during synthetic procedures such as in a substitution or an elimination reaction. Examples of hydroxyl activating groups include, but are not limited to, mesylate, tosylate, triflate, p-nitrobenzoate, phosphonate and the like.

The term “activated hydroxyl,” as used herein, refers to a hydroxy group activated with a hydroxyl activating group, as defined above, including mesylate, tosylate, triflate, p-nitrobenzoate, phosphonate groups, for example.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups are described generally in T. H. Greene and P. G. M. Wuts, Protective Groups in Organic Synthesis, 3rd edition, John Wiley & Sons, New York (1999). Examples of hydroxyl protecting groups include benzyloxycarbonyl, 4-methoxybenzyloxycarbonyl, tert-butoxy-carbonyl, isopropoxycarbonyl, diphenylmethoxycarbonyl, 2,2,2-trichloroethoxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, allyl, benzyl, triphenyl-methyl (trityl), methoxymethyl, methylthiomethyl, benzyloxymethyl, 2-(trimethylsilyl)-ethoxymethyl, methanesulfonyl, trimethylsilyl, triisopropylsilyl, and the like.

The term “pharmaceutically acceptable anion,” as used herein, refers to a conjugate base of a pharmaceutically acceptable acid. Pharmaceutically acceptable acids are acids used to prepare pharmaceutically acceptable acid addition salts, as described, for example, in Stahl, P. H. and Wermuth, C. G. (eds.), Handbook of Pharmaceutical Salts: Properties, Selection and Use, Wiley V C H (2008) and S. M. Berge, et al., J. Pharmaceutical Sciences, 66: 1-19 (1977). Pharmaceutically acceptable anions include, but are not limited to, acetate, dichloroacetate, adipate, alginate, L-ascorbate, L-aspartate, benzenesulfonate, 4-acetamidobenzoate, benzoate, p-bromophenylsulfonate; (+)-camphorate, (+)-camphor-10-sulfonate, caprate, caproate, caprylate, carbonate, cinnamate, cyclamate, dodecylsulfate, ethane-1,2-disulfonate, ethanesulfonate, 2-hydroxyethanesulfonate, sulfate, borate, citrate, formate, fumarate, galactarate, gentisated, D-glucoheptonate, D-gluconate, D-glucuronate, glutamate, glutarate, 2-oxoglutarate, glycerophosphate, glycolate, hippurate, chloride, bromide, iodide, isobutyrate, DL-lactate, lactobionate, laurate, maleate, (−)-L-malate, malonate, DL-mandelate, methanesulfonate, naphthalene-1,5-disulfonate, naphthalene-2-sulfonate, 1-hydroxy-2-naphthoate, nicotinate, nitrate, oleate acid, orotate, oxalate, palmitate, pamoate, phosphate, propionate, (−)-L-pyroglutamate, salicylate, 4-aminosalicylate, sebacyl, stearate, succinate, (+)-L-tartrate, thiocyanate, p-toluenesulfonate, and undecylenate. Preferably, the pharmaceutically acceptable anion is a monoanion. Preferred pharmaceutically acceptable anions include acetate, bromide, camsylate, chloride, formate, fumarate, maleate, mesylate, nitrate, oxalate, phosphate, sulfate, tartrate, thiocyanate and tosylate. Particularly preferred pharmaceutically acceptable anions include chloride, bromide, and acetate.

The term “protected hydroxy,” as used herein, refers to a hydroxy group protected with a hydroxy protecting group, as defined above, including benzoyl, acetyl, trimethylsilyl, triethylsilyl, methoxymethyl groups, for example.

The term “hydroxy prodrug group,” as used herein, refers to a promoiety group which is known in the art to change the physicochemical, and hence the biological properties of a parent drug in a transient manner by covering or masking the hydroxy group. After said synthetic procedure(s), the hydroxy prodrug group as described herein must be capable of reverting back to hydroxy group in vivo. Hydroxy prodrug groups as known in the art are described generally in Kenneth B. Sloan, Prodrugs, Topical and Ocular Drug Delivery, (Drugs and the Pharmaceutical Sciences; Volume 53), Marcel Dekker, Inc., New York (1992) and in “Prodrugs of Alcohols and Phenols” by S. S. Dhareshwar and V. J. Stella, in Prodrugs Challenges and Rewards Part-2, (Biotechnology: Pharmaceutical Aspects), edited by V. J. Stella, et al, Springer and AAPS Press, 2007, pp 31-99.

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the art are described generally in T. H. Greene and P. G. M. Wuts, Protective Groups in Organic Synthesis, 3rd edition, John Wiley & Sons, New York (1999). Examples of amino protecting groups include, but are not limited to, methoxycarbonyl, t-butoxycarbonyl, 9-fluorenyl-methoxycarbonyl, benzyloxycarbonyl, and the like.

The term “protected amino,” as used herein, refers to an amino group protected with an amino protecting group as defined above.

The term “leaving group” means a functional group or atom which can be displaced by another functional group or atom in a substitution reaction, such as a nucleophilic substitution reaction. By way of example, representative leaving groups include chloro, bromo and iodo groups; sulfonic ester groups, such as mesylate, tosylate, brosylate, nosylate and the like; and acyloxy groups, such as acetoxy, trifluoroacetoxy and the like.

The term “aprotic solvent,” as used herein, refers to a solvent that is relatively inert to proton activity, i.e., not acting as a proton-donor. Examples include, but are not limited to, hydrocarbons, such as hexane and toluene, for example, halogenated hydrocarbons, such as, for example, methylene chloride, ethylene chloride, chloroform, and the like, heterocyclyl compounds, such as, for example, tetrahydrofuran and N-methylpyrrolidinone, and ethers such as diethyl ether, bis-methoxymethyl ether. Such compounds are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

The term “protic solvent,” as used herein, refers to a solvent that tends to provide protons, such as an alcohol, for example, methanol, ethanol, propanol, isopropanol, butanol, t-butanol, and the like. Such solvents are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of protogenic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

The term “subject,” as used herein, refers to an animal. Preferably, the animal is a mammal. More preferably, the mammal is a human. A subject also refers to, for example, dogs, cats, horses, cows, pigs, guinea pigs, fish, birds, and the like.

The term “treating”, as used herein, means relieving, lessening, reducing, eliminating, modulating, or ameliorating, i.e., causing regression of the disease state or condition. Treating can also include inhibiting, i.e., arresting the development, of an existing disease state or condition, and relieving or ameliorating, i.e., causing regression of an existing disease state or condition, for example when the disease state or condition may already be present.

The term “preventing”, as used herein means, to completely or almost completely stop a disease state or condition from occurring in a patient or subject, especially when the patient or subject is predisposed to such or at risk of contracting a disease state or condition.

“Solvate” means a solvent addition solid form of a compound that contains either stoichiometric or nonstoichiometric amounts of a solvent. Some compounds tend to trap a fixed molar ratio of solvent molecules in the crystalline solid state, thus forming a solvate. If the solvent is water, the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrates.

### Preparation of Compounds

The compounds and processes of the present invention will be better understood in connection with the following synthetic schemes that illustrate the methods by which the compounds of the invention may be prepared, which are intended as an illustration only and not to limit the scope of the invention. Various changes and modifications to the disclosed embodiments will be apparent to those skilled in the art and such changes and modifications including, without limitation, those relating to the chemical structures, substituents, derivatives, and/or methods of the invention may be made without departing from the spirit of the invention and the scope of the appended claims.

Compounds 1, 2, 3, 6 and 7 are prepared by reacting commercially available Compound C with the appropriate alkyl halide or substituted alkyl halide as shown in Scheme 1 below. The reaction is conducted in a polar solvent such as dimethylsulfoxide.

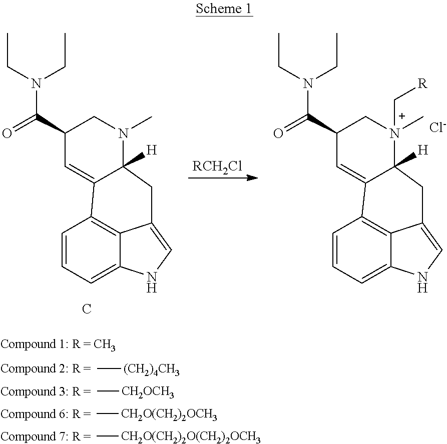

Compounds 4 and 5 are prepared as shown in Scheme 2 below. The first reaction is conducted in a polar solvent such as dimethylsulfoxide.

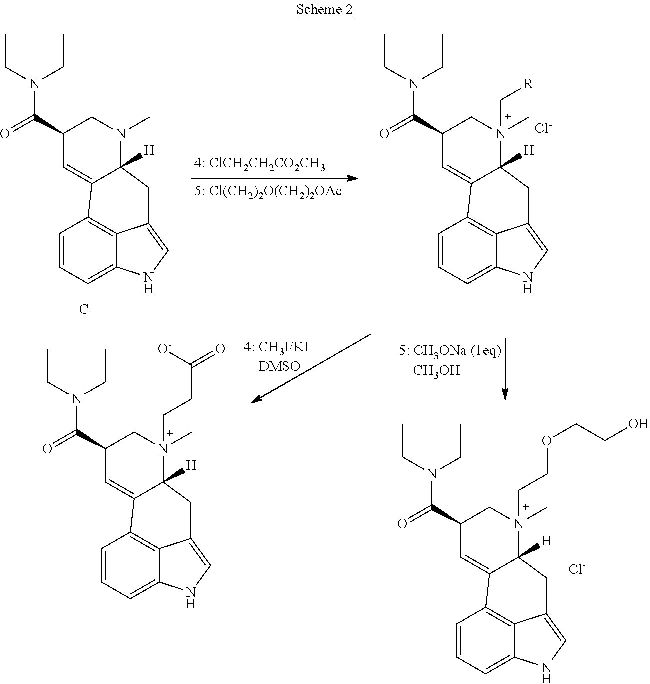

Compounds 8, 9, and 11 are prepared by reacting commercially available Compound D with a suitable alkyl chloride as shown in Scheme 3 below. The reaction is conducted in a polar solvent such as dimethylsulfoxide.

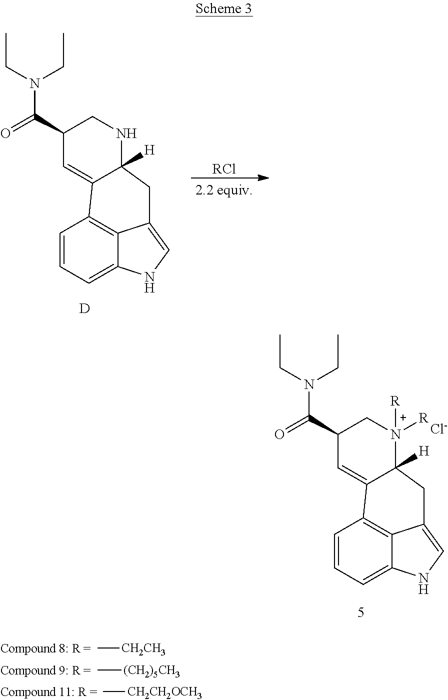

Compound 10 is prepared as shown in Scheme 4 below. The reaction is conducted in a polar solvent such as dimethylsulfoxide and proceeds via slow addition of 1,4-dichlorobutane to Compound D.

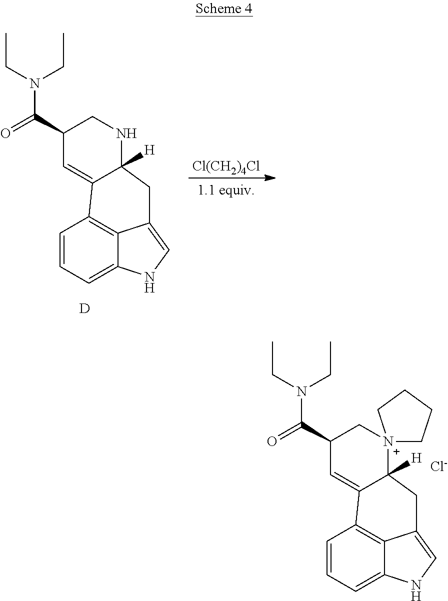

Schemes 1-4 exemplify the use of alkyl chlorides and substituted alkyl chlorides, but the corresponding bromides and iodides can also be used.

The synthesized compounds can be separated from a reaction mixture and further purified by a method such as column chromatography, high pressure liquid chromatography, or recrystallization. As can be appreciated by the skilled artisan, further methods of synthesizing the compounds of the invention herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2nd Ed. Wiley-V C H (1999); T. W. Greene and P. G. M. Wuts, Protective Groups in Organic Synthesis, 3rd Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The above schemes illustrate the preparation of chloride salts of Compounds 1-3 and 5-11. Other salts of these compounds can be obtained using anion exchange methods known in the art.

The biological activity of the compounds of the invention is evaluated using methods known in the art. Changes in morphology, gene expression, epigenetic and electrophysiological activity among DRG neurons after experimental peripheral nerve injury and in rodent models of diabetic neuropathy and other experimental models of neuropathic pain including chemotherapy-induced peripheral neuropathy (CIPN) (Yeh T Y, et al., Cells. 2020 Dec. 21; 9 (12): 2725). Characterization of these phenomena and their responsiveness to therapeutic intervention are used to understand the effects of experimental drugs on maladaptive functional and sensory changes that underlie neuropathic pain in humans. Structural (morphological) changes modeled in rodent DRGs include differences in the numbers of neurons that express β3-tubulin, a reduction in the numbers of neurite-expressing cultured DRG neurons, and defects in neurite outgrowth and branching in vitro (De Gregorio C, et al., Dis Model Mech. 2021 Jan. 1; 14 (1): dmm046334). Changes in the receptor expression profiles of DRG cells after nerve injury include a decrease in CGRP-expressing neurons and an increase in P2X3+ and ATF3+ neurons, such as seen in rodent models of CIPN (Yeh T Y, et al Cells. 2020 Dec. 21; 9 (12): 2725). Changes in the regulation of sensory neuron genes including ATF3, BDNF, galanin, and neuropeptide Y (NPY) are used to characterize neuropathic-pain related sensory neuron and macrophage function in vitro (Yu X, et al Nat Commun. 2020 Jan. 14; 11 (1): 264). Similarly, epigenetic modifications that occur following nerve injury (Penas C, et al. Front Cell Neurosci. 2018 Jun. 7; 12:158.) can be used to predict the potential therapeutic effects of novel compounds. Alterations in electrophysiological activity of sensory neurons that occur following nerve injury are another approach for experimentally characterizing nerve injury and the potential beneficial effects of therapeutic interventions (Geramifard N, et al., Micromachines (Basel). 2022 Oct. 8; 13 (10): 1692). In addition to exploring changes in the morphology, gene expression, epigenetic activity and electrophysiology of rodent DRG neurons, human induced pluripotent stem cell (iPSC) derived sensory (DRG) neurons can be similarly studied, providing an additional measure of clinical validity (Li R, et al., Front Cell Dev Biol. 2021 Dec. 14; 9:796960).

Rodent behavioral readouts are used to characterize a wide range of phenomena including neuropathic and inflammatory pain, the reversal of pain by analgesics, and the differentiation between analgesia and sedation (Zhang Z, et al., Pain. 2022 Dec. 1; 163 (12): 2326-2336). Behavioral approaches relevant to analgesic development include changes in reflexive withdrawal thresholds to thermal and mechanical stimuli (Modi A D et al Behav Brain Res. 2023 May 28; 446:114417), plantar mechanical hypersensitivity, facial expressions (Klassen-Ross T, et al., Nat Methods. 2010 June; 7 (6): 447-9), and voluntary behaviors such as operant behavior, wheel running, burrowing, nesting, and gait (Tappe-Theodor A, et al., Neurosci Biobehav Rev. 2019 May; 100:335-343). Additionally, spontaneous pain-related rodent behaviors such as flinching, biting, grooming, rearing, ambulation, and time standing still can be quantified using bottom-up behavioral screening (Roberson, David P. et al., Automated Detection of Mouse Pain Behavioral Readouts by Alternating Bottom-Up Pose and Paw Contact Measurements. Available at http://dx.doi.org/10.2139/ssrn.3793964).

Rodent behaviors can also be used to characterize the presence of hallucinations and other psychedelic-related effects, as shown by the mouse and rat head twitch response (HTR) and ultrasonic vocalizations (Jefferson S J, et al., Neuropsychopharmacology. 2023 Apr. 4).

Unless otherwise defined, all technical and scientific terms used herein are accorded the meaning commonly known to one with ordinary skill in the art. All publications, patents, published patent applications, and other references mentioned herein are hereby incorporated by reference in their entirety.

### EXAMPLES

### Example 1 Synthesis of Intermediate Compounds

### (a) Synthesis of (6aR,9R)-7-methyl-4,6,6a, 7,8,9-hexahydroindolo[4,3-fg]quinoline-9-carboxylic acid (Compound B)

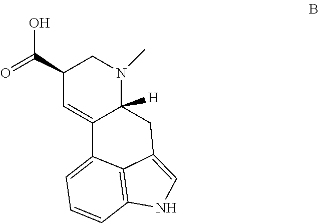 [SMILES: CN1C[C@H](C(=O)O)C=C2c3cccc4[nH]cc(c34)C[C@H]21]

To a solution of commercial d-lysergic acid methyl ester (Compound A, 4.59 g, 16.3 mmol, 1 eq) in ethanol (163 mL) at room temperature was slowly added aqueous 1M NaOH (163 mL, 163 mmol, 10 eq). The reaction was heated at 40° C. for 2 h. After cooling to room temperature, the reaction mixture was acidified with aqueous HCl (2M) to pH 6, partially concentrated, and ice-water added (50 mL). The resulting solid was collected by filtration, washed with cold water, and dried under vacuum to afford 2 (3.14 g, 72%) as a brown solid. The crude material was used without further purification in the next step. ¹H NMR (400 MHz, DMSO-d₆, ppm): δ 10.68 (s, 1H), 7.15, (d, J=7.1 Hz, 1H), 7.04-7.00 (m, 3H), 6.42 (s, 1H), 3.49-3.39 (m, 2H), 3.10 (dd, J=11.2, 5.0 Hz, 1H), 2.99-2.93 (m, 1H), 2.43 (s, 3H).

### (b) Synthesis of (6aR,9R)—N,N-diethyl-7-methyl-4,6,6a,7,8,9-hexahydroindolo[4,3-fg]quinoline-9-carboxamide (C)

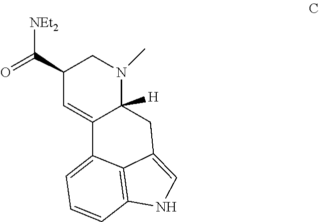

To a solution of 2 (3.14 g, 11.7 mmol, 1 eq) in anhydrous DMF (60 mL) was added CDI (2.85 g, 17.6 mmol, 1.5 eq) at room temperature under N₂ atmosphere. After 1 h, diethylamine (12.1 mL, 117 mmol, 10 eq) was slowly added. After stirring overnight, the reaction was cooled to 0° C. and water (50 mL) slowly added. The mixture was poured into EtOAc (500 mL) and washed with water (3×250 mL) then brine (100 mL). The organic phase was dried over anhydrous Na₂SO₄ and concentrated. The crude material was purified by silica gel flash chromatography (DCM to 90:10:1 DCM/MeOH/NH₄OH) to afford 3 (2.41 g, 64%) as a tan solid. ¹H NMR (400 MHz, CDCl₃, ppm): δ 7.95 (s, 1H), 7.21-7.13 (m, 3H), 6.90 (s, 1H), 6.34 (s, 1H), 3.90-3.83 (m, 1H), 3.55 (dd, J=14.4, 5.5 Hz, 1H), 3.49-3.40 (m, 4H), 3.25-3.19 (m, 1H), 3.05 (dd, J=11.2, 4.8 Hz, 1H), 2.89 (t, J=10.8 Hz, 1H), 2.67 (t, J=12.9 Hz, 1H), 2.59 (s, 3H), 1.23 (t, J=7.1 Hz, 3H), 1.16 (t, J=7.0 Hz, 3H).

### Example 2 Synthesis of (6aR,9R)-9-(diethylcarbamoyl)-7-ethyl-7-methyl-4,6,6a,7,8,9-hexahydroindolo[4,3-fg]quinolin-7-ium Iodide (Compound 1 Iodide Salt)

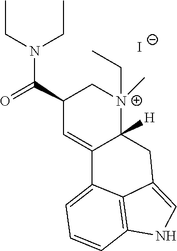 [SMILES: CCN(CC)C(=O)[C@@H]1C=C2c3cccc4[nH]cc(c34)C[C@H]2[N+](C)(CC)C1 ; [I-]]

To a solution of Compound C (300 mg, 0.928 mmol, 1 eq) in DMSO (0.93 mL) was added iodoethane (82 μL, 1.02 mmol, 1.1 eq) in a sealed vial and reaction heated at 50° C. overnight. The crude reaction mixture was purified by C18 reverse phase flash chromatography (H₂O to MeCN) to afford Compound 1 as the iodide salt (78 mg, 18%, 55:45 dr by 1H NMR) as a tan solid. ¹H NMR (400 MHz, CD₃CN, ppm): δ 9.43 (s, 1H), 7.36-7.33 (m, 1H), 7.25 (d, J=7.3 Hz, 1H), 7.17-7.12 (m, 1H), 7.09 (s, 1H), 6.54 (s, 1H), 4.74-4.56 (m, 1H, diastereotopic), 4.05-3.06 (m, 14H), 1.38 (t, J=6.9 Hz, 3H), 1.26 (m, 3H), 1.11 (t, J=7.1 Hz, 3H). MS(ESI+): m/z 352.2.

### Example 3 Synthesis of (6aR,9R)-9-(diethylcarbamoyl)-7-hexyl-7-methyl-4,6,6a, 7,8,9-hexahydroindolo[4,3-fg]quinolin-7-ium Iodide (Compound 2 Iodide Salt)

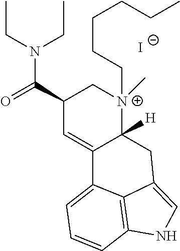 [SMILES: CCCCCC[N+]1(C)C[C@H](C(=O)N(CC)CC)C=C2c3cccc4[nH]cc(c34)C[C@H]21 ; [I-]]

To a solution of Compound C (300 mg, 0.928 mmol, 1 eq) in DMSO (0.93 mL) was added iodohexane (0.15 mL, 1.02 mmol, 1.1 eq) in a sealed vial and reaction heated at 50° C. overnight. The crude reaction mixture was purified by C18 reverse phase flash chromatography (H₂O to MeCN) to afford Compound 2 as the iodide salt (30 mg, 6%) as a beige solid. ¹H NMR (400 MHz, CD₃CN, ppm): δ 9.33 (s, 1H), 7.34 (d, J=8.0 Hz, 1H), 7.26 (d, J=7.3 Hz, 1H), 7.16 (t, J=7.8 Hz, 1H), 7.10 (s, 1H), 6.55 (s, 1H), 4.64-4.58 (m, 1H), 4.05-3.98 (m, 1H), 3.81 (t, J=11.7 Hz, 1H), 3.74-3.41 (m, 7H), 3.36-3.27 (m, 1H), 3.16-3.08 (m, 4H), 1.86-1.75 (m, 2H), 1.40-1.30 (m, 6H), 1.26 (t, J=7.1 Hz, 3H), 1.11 (t, J=7.1 Hz, 3H), 0.88 (t, J=6.6 Hz, 3H). MS(ESI+): m/z 408.3

### Example 4 Synthesis of (6aR,9R)-9-(diethylcarbamoyl)-7-methyl-4,6,6a, 7,8,9-hexahydroindolo[4,3-fg]quinoline 7-oxide (Compound 15)

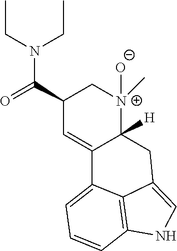 [SMILES: CCN(CC)C(=O)[C@@H]1C=C2c3cccc4[nH]cc(c34)C[C@H]2[N+](C)([O-])C1]

To a solution of Compound C (410 mg, 1.27 mmol, 1 eq) in DCM (5.1 mL) at 0° C. was slowly added mCPBA (75% by wt.) (292 mg, 1.27 mmol, 1 eq) in DCM (2.5 mL) under N₂ atmosphere. After 1 h, reaction mixture poured into 1M NaOH (50 mL) and extracted with DCM (3×30 mL). The combined organic phase was washed with brine (15 mL), dried over anhydrous Na₂SO₄, and concentrated. The crude material was purified by silica gel flash chromatography (DCM to 90:10:1 DCM/MeOH/NH₄OH) to afford Compound 15 (150 mg, 35%) as a beige solid. ¹H NMR (400 MHz, CDCl₃, ppm): δ 9.61 (s, 1H), 7.16 (d, J=7.8 Hz, 1H), 7.10-7.02 (m, 2H), 6.72 (s, 1H), 6.35 (s, 1H), 4.52-4.48 (m, 1H), 4.08-4.04 (m, 1H), 3.82 (t, J=11.1 Hz, 1H), 3.72-3.66 (m, 1H), 3.60 (dd, J=11.2, 4.6 Hz, 1H), 3.45-3.15 (m, 8H), 1.14-1.08 (m, 6H). MS(ESI+): m/z 340.2.

### Example 5 Synthesis of (6aR,9R)-9-(diethylcarbamoyl)-7-(4-ethoxy-4-oxobutyl)-7-methyl-4,6,6a, 7,8,9-hexahydroindolo[4,3-fg]quinolin-7-ium Bromide (Compound 14 Bromide Salt)

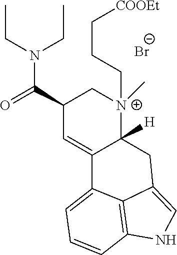 [SMILES: CCOC(=O)CCC[N+]1(C)C[C@H](C(=O)N(CC)CC)C=C2c3cccc4[nH]cc(c34)C[C@H]21 ; [Br-]]

To a solution of Compound 3 (300 mg, 0.928 mmol, 1 eq) in DMSO (0.93 mL) was added ethyl 4-bromobutyrate (198 mg, 1.02 mmol, 1.1 eq) in a sealed vial and reaction heated at 50° C. overnight. The crude reaction mixture was purified by C18 reverse phase flash chromatography (H₂O to MeCN) to afford Compound 14 as the bromide salt (26 mg, 5%) as a yellow solid. ¹H NMR (400 MHz, CD₃CN, ppm): δ 10.04 (s, 1H), 7.35 (d, J=8.0 Hz, 1H), 7.21 (d, J=7.3 Hz, 1H), 7.11 (t, J=7.7 Hz, 1H), 7.05 (s, 1H), 6.52 (s, 1H), 4.62-4.58 (m, 1H), 4.12-4.04 (m, 3H), 3.89-3.72 (m, 3H), 3.66-3.42 (m, 5H), 3.33-3.26 (m, 1H), 3.18 (s, 3H), 3.05 (t, J=13.1 Hz, 1H), 2.49-2.35 (m, 2H), 2.11-2.01 (m, 2H), 1.27 (t, J=7.1 Hz, 3H), 1.20 (t, J=7.1 Hz, 3H), 1.10 (t, J=7.1 Hz, 3H). MS(ESI+): m/z 438.3.

### Example 6 Synthesis of (6aR,9R)-9-(diethylcarbamoyl)-7-isobutyl-7-methyl-4,6,6a, 7,8,9-hexahydroindolo[4,3-fg]quinolin-7-ium Iodide (Compound 12 Iodide Salt)

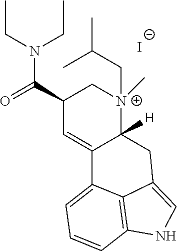 [SMILES: CCN(CC)C(=O)[C@@H]1C=C2c3cccc4[nH]cc(c34)C[C@H]2[N+](C)(CC(C)C)C1 ; [I-]]

To a solution of Compound C (300 mg, 0.928 mmol, 1 eq) in DMSO (0.93 mL) was added 1-iodo-2-methylpropane (0.12 mL, 1.02 mmol, 1.1 eq) in a sealed vial and reaction heated at 50° C. overnight. The crude reaction mixture was purified by C18 reverse phase flash chromatography (H₂O to MeCN) to afford Compound 12 as the iodide salt (42 mg, 9%, 55:45 dr by 1H NMR) as a yellow solid. ¹H NMR (400 MHz, CD₃CN, ppm): δ 9.25 (s, 1H), 7.35-7.32 (m, 1H), 7.28 (t, J=7.2 Hz, 1H), 7.17 (t, J=7.8 Hz, 1H), 7.10 (s, 1H), 6.57 (m, 1H, diastereotopic), 4.68-4.59 (m, 1H), 4.04-3.11 (m, 14H), 2.39-2.31 (m, 1H), 1.28-1.24 (m, 3H), 1.20-1.08 (m, 9H). MS(ESI+): m/z 380.2.

### Example 7 Synthesis of (6aR,9R)-7-(4-(tert-butoxy)-4-oxobutyl)-9-(diethylcarbamoyl)-7-methyl-4,6,6a,7,8,9-hexahydroindolo[4,3-fg]quinolin-7-ium Bromide (Compound 16 Bromide Salt)

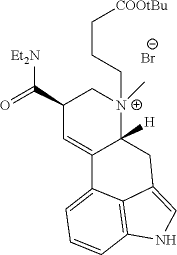

To a solution of Compound C (800 mg, 2.47 mmol, 1 eq) in DMSO (2.5 mL) was added t-butyl 4-bromobutanoate (607 mg, 2.72 mmol, 1.1 eq) in a sealed vial and reaction heated at 50° C. overnight. The crude reaction mixture was purified by C18 reverse phase flash chromatography (H₂O to MeCN) to afford Compound 16 as the bromide salt (119 mg, 9%) as a yellow solid. ¹H NMR (400 MHz, CD₃CN, ppm): δ 10.10 (s, 1H), 7.35 (d, J=7.8 Hz, 1H), 7.21 (d, J=7.3 Hz, 1H), 7.12 (t, J=7.8 Hz, 1H), 7.06 (s, 1H), 6.52 (s, 1H), 4.64-4.59 (m, 1H), 4.08-4.03 (m, 1H), 3.87-3.42 (m, 8H), 3.30-3.26 (m, 1H), 3.18 (s, 3H), 3.06 (t, J=13.1 Hz, 1H), 2.39-2.25 (m, 2H), 2.09-1.98 (m, 2H), 1.42 (s, 9H), 1.27 (t, J=7.1 Hz, 3H), 1.10 (t, J=7.0 Hz, 3H).

### Example 8 Synthesis of (6aR,9R)-7-(3-carboxypropyl)-9-(diethylcarbamoyl)-7-methyl-4,6,6a,7,8,9-hexahydroindolo[4,3-fg]quinolin-7-ium Chloride (Compound 13 Chloride Salt)

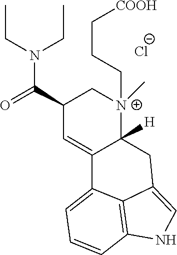 [SMILES: CCN(CC)C(=O)[C@@H]1C=C2c3cccc4[nH]cc(c34)C[C@H]2[N+](C)(CCCC(=O)O)C1 ; [Cl-]]

To a solution of Compound 16 bromide salt (119 mg, 0.218 mmol, 1 eq) in anhydrous 1,4-dioxane (0.55 mL) was added HCl (4M in dioxane) (0.55 mL, 2.18 mmol, 10 eq) in a sealed vial at room temperature. After 2 h, the reaction mixture was concentrated. The crude reaction mixture was purified by C18 reverse phase flash chromatography (H₂O to MeCN) to afford Compound 13 as the chloride salt (10 mg, 10%) as a beige solid.
¹H NMR (400 MHz, D₂O, ppm): δ 7.15 (d, J=8.0 Hz, 1H), 6.90 (t, J=7.7 Hz, 1H), 6.83 (s, 1H), 6.74 (d, J=7.3 Hz, 1H), 5.90 (s, 1H), 3.67-3.56 (m, 2H), 3.48-3.10 (m, 8H), 2.93-2.84 (m, 1H), 2.59 (s, 3H), 2.43 (t, J=12.8 Hz, 1H), 2.03 (t, J=6.5 Hz, 2H), 1.69-1.56 (m, 2H), 1.17 (t, J=6.9 Hz, 3H), 1.01 (t, J=6.9 Hz, 3H). MS(ESI+): m/z 410.2.

### Example 9 In Vitro Evaluation of Compound 1 and Compound 2

Methods: Assessment of Compound 1 Induced Calcium Flux in Cultured DRG Neurons To evaluate the effect of Compound 1 and Compound 2 on calcium flux in cultured dorsal root ganglion (DRG) neurons isolated from 8-week-old male mice, the following protocols were performed:

### Materials and Reagents

8-week-old C57BL6 male mice
Fura-2AM (calcium indicator dye)
Compound 1 (at concentrations of 1 μM and 10 μM)
Compound 2 (at concentrations of 1 μM and 10 μM)
ATP (P2X3R agonist, 10 μM)
Capsaicin (TRPV1 ligand, 10 μM)
KCl (50 mM)
Calcium imaging setup (calcium microscope)

### Procedure

### 1. Animal Preparation and DRG Harvesting

Euthanize 8-week-old male mice according to ethical guidelines.
Harvest the dorsal root ganglia (DRG) from the mice.

### 2. Cell Culture

Culture the DRG neurons in appropriate growth media for 24 hours.
   3. Loading Neurons with Fura-2AM
Load the cultured DRG neurons with Fura-2AM to enable calcium imaging.
Incubate the neurons with Fura-2AM for the required duration as per manufacturer's instructions.

### 4. Calcium Imaging

Set up the calcium microscope for imaging single-neuron calcium flux.
Sequentially expose the neurons to the following compounds and record calcium flux at specified time intervals:

### Compound 1 Protocol:

Compound 1 (1 μM): Apply for 60-90 seconds.
Compound 1 (10 μM): Apply for 240-270 seconds.
ATP (P2X3R agonist, 10 μM): Apply for 480-510 seconds.
Capsaicin (TRPV1 ligand, 10 μM): Apply for 720-735 seconds.
KCl (pan-neuron activator, 50 mM): Apply for 960-975 seconds.

### Compound 2 Protocol

Compound 2 (1 μM): Apply for 60-90 seconds.
Compound 2 (10 μM): Apply for 240-270 seconds.
ATP (P2X3R agonist, 10 μM): Apply for 480-510 seconds.
Capsaicin (TRPV1 ligand, 10 μM): Apply for 720-735 seconds.
KCl (50 mM): Apply for 960-975 seconds.

### 5. Data Analysis

Measure and record the average calcium flux (A).
Determine the percentage of responding neurons, identifying responding neurons as those with an f340/f380 fluorescence ratio >10% (B).

### 6. Results

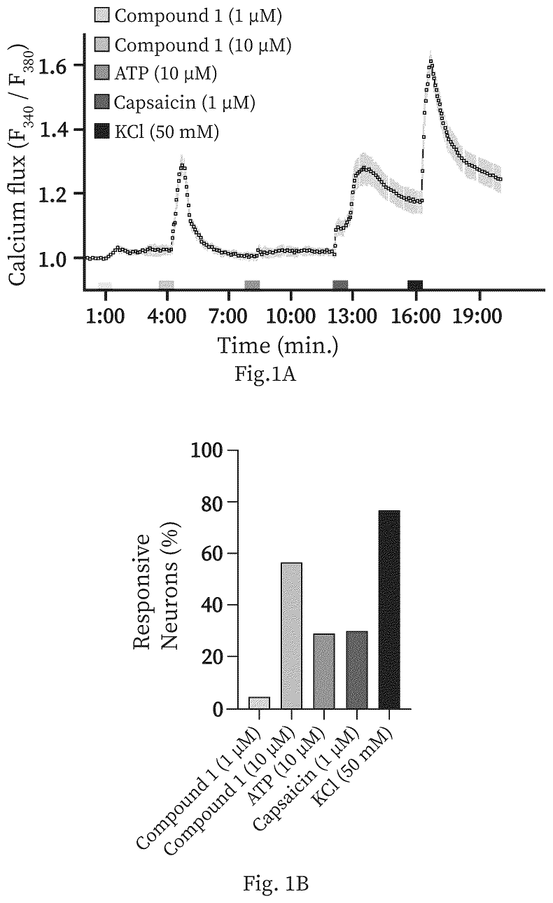

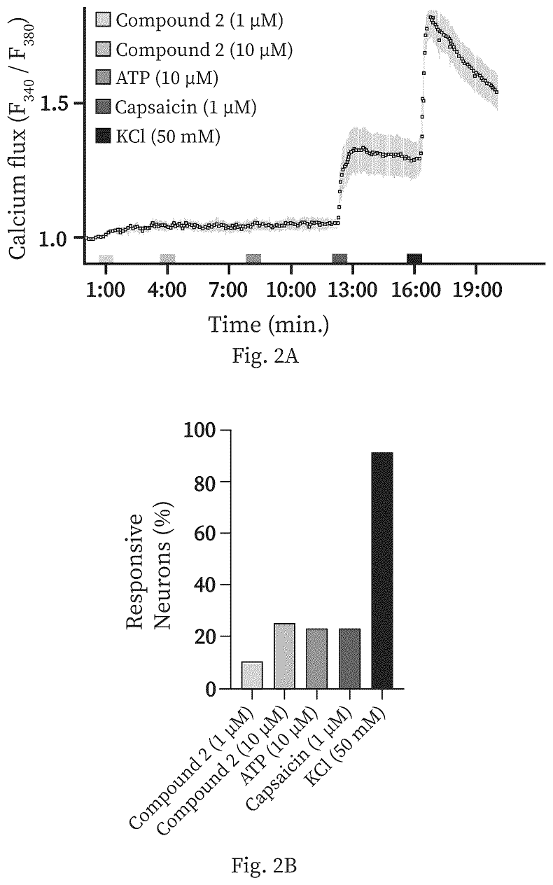

Compound 1 (10 μM) induces calcium flux in approximately 60% of KCl-responsive neurons; n=3 mice, 110 neurons.
Compound 2 induces calcium flux at 10 μM in approximately 20% of KCl-responsive neurons; n=3 mice, 100 neurons.
   The data are presented in graphical format in FIGS. 1A and 1B (Compound 1) and FIGS. 2A and 2B (Compound 2), showing both average calcium flux and percentage of responding neurons.

Proper calibration of the calcium imaging setup was ensured before starting the experiments. All reagents and mice were handled according to institutional guidelines and safety protocols. All biological waste was disposed of following appropriate biohazard disposal procedures.

Eight-week-old C57BL6 male mice were euthanized, DRG harvested, and neurons cultured for 24 hours. The neurons were then loaded with Fura-2AM, a calcium-responsive dye, and single-neuron calcium flux was recorded by calcium microscopy. The neurons were then sequentially exposed to Compound 1 (1 μM, 60-90 sec), Compound 1 (10 μM, 240-270 sec), ATP (P2X3R agonist; 10 μM, 480-510 sec), capsaicin (TRPV1 ligand; 10 μM, 720-735 sec), and KCl (pan-neuron activator; 50 mM, 960-975 sec). Average calcium flux (FIG. 1A) and the percentage (FIG. 1B) of responding neurons (identified as f340/f380>10% fluorescence) are shown. Compound 1 (10 μM) induced calcium flux in ˜60% of exposed KCl+ neurons. n=3 mice; 110 neurons.

Eight-week-old C57BL6 male mice were euthanized, DRG harvested, and neurons cultured for 24 hours. The neurons were then loaded with Fura-2AM, a calcium-responsive dye, and single-neuron calcium flux was recorded by calcium microscopy. The neurons were then sequentially exposed to Compound 2 (1 μM, 60-90 sec), Compound 2 (10 μM, 240-270 sec), ATP (P2X3R agonist; 10 μM, 480-510 sec), capsaicin (TRPV1 ligand; 10 μM, 720-735 sec), and KCl (pan-neuron activator; 50 mM, 960-975 sec). Average calcium flux (FIG. 2A) and the percentage (FIG. 2B) of responding neurons (identified as f340/f380>10% fluorescence) are shown. Compound 2 (10 μM) induced calcium flux in ˜20% of exposed KCl+ neurons. n=3 mice; 100 neurons.

### Example 10 In Vivo Evaluation of Compounds 1 and 2

### Methods: Assessment of Pain Response in Mice Using Hargreaves Test and Blackbox Behavioral Analysis

To evaluate the effect of different treatments on voluntary pain-related behaviors in mice, we used the Blackbox behavioral analysis system, as previously described (Zhang, et al., PAIN, 2022 Dec. 1; 163 (12): 2326-2336, incorporated herein by reference in its entirety).

### Materials and Reagents

Male and female C57BL6 mice (n=5 per group)
Complete Freund's Adjuvant (CFA, Sigma)
Saline
PBS
DMSO
Compound 1
Compound 2
Syringes (for injections)
Hargreaves apparatus (for thermal pain testing)
Blackbox behavioral analysis setup

### Procedure

### 1. Animal Preparation and Injection

Divide mice into groups (n=10 per group).
Inject 20 μL of CFA or saline into the right hind paw of each mouse.

### 2. Grouping and Drug Administration

24 hours after CFA injection, randomly assign mice to the following groups:
Control Group: PBS+PBS
CFA+PBS Group
CFA+DMSO Group
CFA+Compound 1 Group
CFA+Compound 2 Group

### 3. Drug Injection

Inject the designated treatment (PBS (20 μL), DMSO (20 μL), Compound 1 (1 μM in 20 μL PBS), or Compound 2 (1 μM in 20 μL DMSO)) into the plantar right paw of each mouse according to their assigned group.

### 4. Behavioral Testing Schedule

Perform Blackbox behavioral analysis and Hargreaves test at the following time points after drug injection: 1 hour, 3 hours, 6 hours, and 24 hours.

### 5. Behavioral Testing Procedures

Blackbox Behavioral Analysis:
Place each mouse in the Blackbox apparatus for 10 minutes.
Record and analyze behaviors indicative of pain or discomfort (e.g., grooming, paw licking, flinching).

### 6. Data Collection and Analysis

Record the data for each mouse at each time point.
Compare the results across different groups to assess the effects of the treatments on pain response.

### Notes

All procedures were performed in compliance with institutional guidelines for animal care and use. Mice were handled gently to minimize stress and variability in behavioral responses.

### SUMMARY

This protocol outlines the steps to assess the impact of different treatments on pain response in mice using Blackbox behavioral analysis. Mice are injected with CFA or saline, followed by treatment with Compound 1 or Compound 2, and negative controls using vehicle (PBS or DMSO) alone.

### Groups and Treatments:

1. Control Group: PBS+PBS
2. CFA+PBS Group
3. CFA+DMSO Group
4. CFA+Compound 1 Group
5. CFA+Compound 2 Group

### Behavioral Testing Time Points:

1 hour after drug injection
3 hours after drug injection
6 hours after drug injection
24 hours after drug injection
   Precise timing and consistent handling throughout the experiment were utilized to maintain the validity of the results.

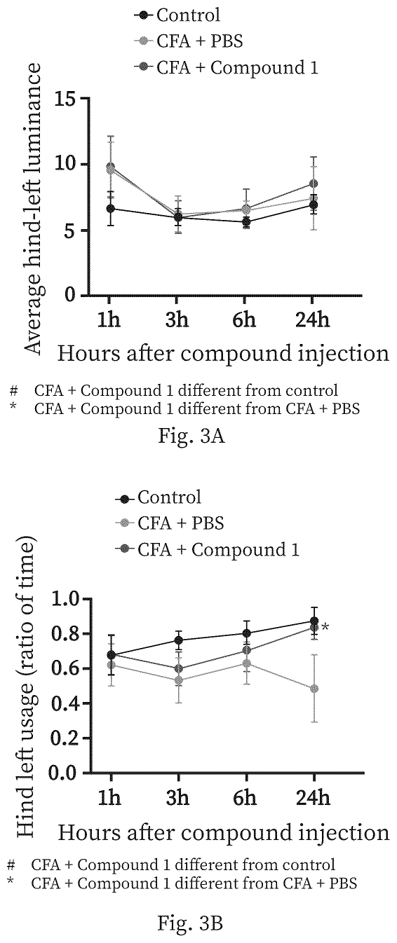

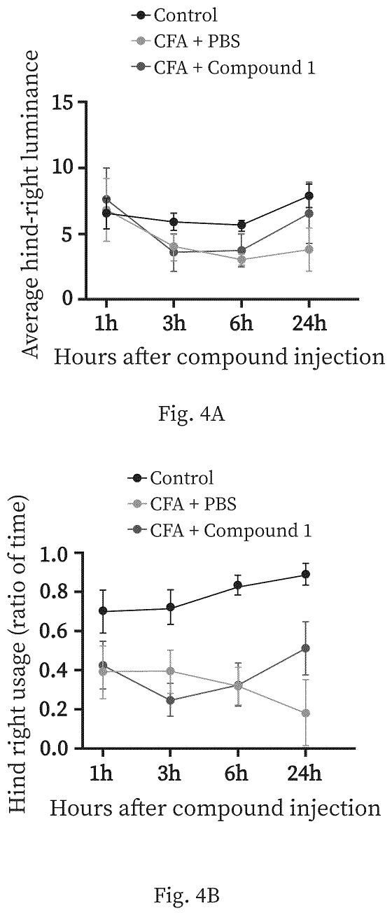

Effect of Compound 1. Eight-week-old male and female mice (n=5 mice/group) were injected with CFA (20 μL; intradermal) or saline (20 μL) into the right hind paw. Left (contralateral) hindpaw luminance (FIG. 3A) and ratio of time (FIG. 3B), as well as right hindpaw luminance (FIG. 4A) and ratio of time (FIG. 4B), were assessed as surrogates for mechanical pain hypersensitivity at 1-, 3-, 6-, and 24-hours post-treatment. Compared to control mice, CFA in the right paw did not change left paw usage with Compound 1 treatment or with PBS (vehicle) treatment. CFA in the right paw durably reduced the right paw usage of these mice. This effect was partly reduced by Compound 1 treatment.

Data were analyzed using a two-way ANOVA to assess the effects of CFA injection and subsequent treatments on hindpaw usage and thermal pain hypersensitivity. Post-hoc comparisons were performed using Dunnett's test to identify significant differences between treatment groups.

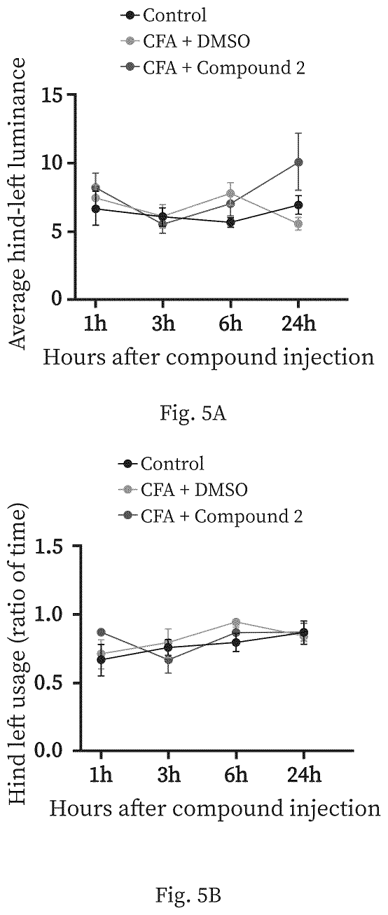

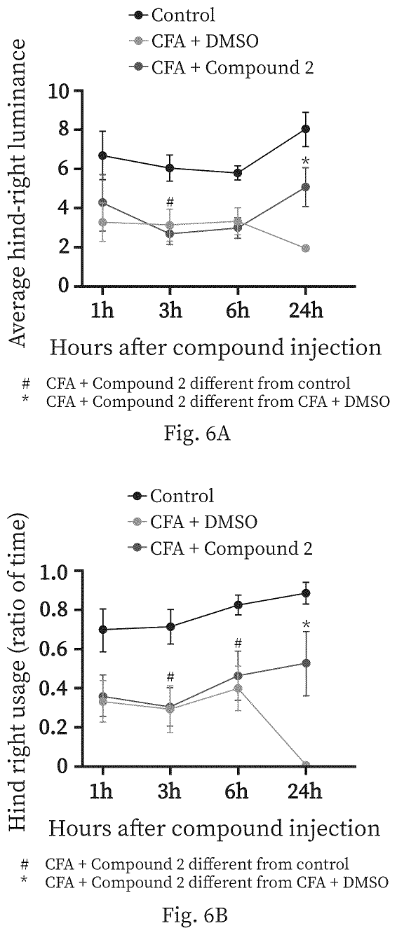

Effect of Compound 2. Eight-week-old male and female mice (n=5 mice/group) were injected with CFA (20 μL; intradermal) or saline (20 μL) into the right hind paw. Left (contralateral) hind paw luminance (FIG. 5A) and ratio of time (FIG. 5B), as well as right hindpaw luminance (FIG. 6A) and ratio of time (FIG. 6B), were assessed as a readout of mechanical pain hypersensitivity at 1-, 3-, 6-, and 24-hours post-treatment. Compared to control mice, CFA in the right paw did not change left paw usage with Compound 2 treatment or with DMSO (vehicle) treatment. Compared to vehicle-exposed mice, CFA durably reduced the right (injured) paw usage of these mice. This effect was partly reduced by Compound 2 treatment beginning 24 hours after compound administration.

Data were analyzed using a two-way ANOVA to assess the effects of CFA injection and subsequent treatments on hindpaw usage (mechanical hypersensitivity). Post-hoc comparisons were performed using Dunnett's test to identify significant differences between treatment groups.

### Interpretation of In Vivo Experimental Results:

Both Compound 1 and Compound 2 produced partial reversal of CFA-induced mechanical hypersensitivity beginning at 24 hours after intraplantar administration. Together with the calcium imaging results, showing activation of DRG neurons by COMPOUND 1 but not by Compound 2, these data support the utility of these compounds for treatment of inflammatory pain in a mouse chronic pain model. The ability of both Compound 1, which produced calcium flux, and Compound 2 which did not produce calcium flux, to reverse pain behaviors beginning 24 hours after administration suggests that the analgesic effect does not require activation of extracellular receptors expressed on DRG cells, suggesting that the analgesic effect may arise from activation of intracellular 5-HT2A receptors secondary to cell entry via TRPV1 channels, or due to an effect of the compounds on immune cells to produce an anti-inflammatory response.

While this invention has been particularly shown and described with reference to preferred embodiments thereof, it will be understood by those skilled in the art that various changes in form and details may be made therein without departing from the scope of the invention encompassed by the appended claims.

1. A compound represented by Formula (I), 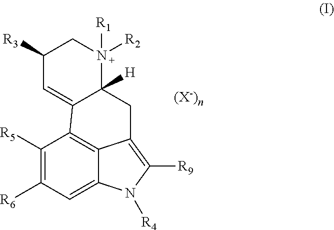
wherein
R₁ and R₂ are independently selected from the group consisting of substituted or unsubstituted C₁-C₁₂-alkyl, C₂-C₁₂-alkenyl; —CH₂CH₂—(OCH₂CH₂)ₘOR₁₂; and C₁-C₁₂-alkyl-Y; or
one of R₁ and R₂ is —O⁻; or
R₁ and R₂ are taken together with nitrogen atom to which they are attached to form a substituted or unsubstituted 3- to 8-membered heterocyclyl;
R₃ is selected from the group consisting of hydrogen, halogen, substituted or unsubstituted C₁-C₆-alkyl, —OR₈, —NR₇R₈, —C(O)NR₇R₈, —C(O)O⁻, and —C(O)OR₈;
R₄ is selected from the group consisting of substituted or unsubstituted C₁-C₆-alkyl, C₁-C₆-alkyl-OR₁₀, C₁-C₆-alkyl-NR₁₀R₁₁, —C(O)R₁₀, and —C(O)NR₁₀R₁₁;
R₅ and R₆ are independently selected from the group consisting of hydrogen, halogen, hydroxyl, optionally substituted C₁-C₆-alkyl and C₁-C₆-alkoxy;
R₇ and R₈ are independently selected from the group consisting of hydrogen and substituted or unsubstituted C₁-C₆-alkyl; or R₇ and R₈ are taken together with nitrogen atom to which they are attached to form a substituted or unsubstituted 3- to 8-membered heterocyclyl;
R₉ is selected from the group consisting of hydrogen, halogen, and substituted or unsubstituted C₁-C₆-alkyl;
R₁₀ and R₁₁ are independently selected from the group consisting of hydrogen and C₁-C₆-alkyl;
R₁₂ is hydrogen or substituted or unsubstituted C₁-C₆-alkyl;
Y is selected from the group consisting of hydroxyl, substituted or unsubstituted C₁-C₆-alkoxy; substituted or unsubstituted C₁-C₆-alkyl-OC(O)—; and —C(O)O⁻;
X⁻ is a pharmaceutically acceptable anion;
m is an integer from 1 to 5;
n is 0 when at least one of R₃ or Y is —C(O)O⁻; and
n is 1 when neither R₃ nor Y is —C(O)O⁻;
provided that the compound is not

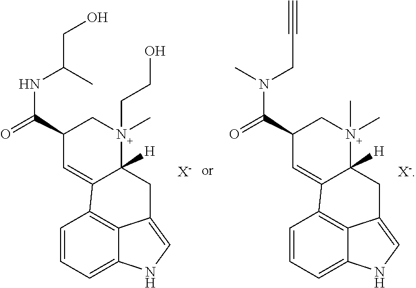

2. The compound of claim 1, represented by Formula (II),

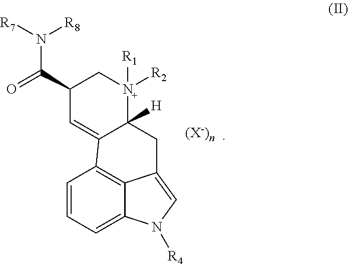

3. The compound of claim 2 wherein R₇ and R₈ are independently C₁-C₆-alkyl.

4. (canceled)

5. The compound of claim 1 wherein R₁ and R₂ are independently C₁-C₁₂-alkyl or C₁-C₁₂-alkyl-Y.

6-13. (canceled)

14. The compound of claim 1 which is selected from the compounds set forth below:

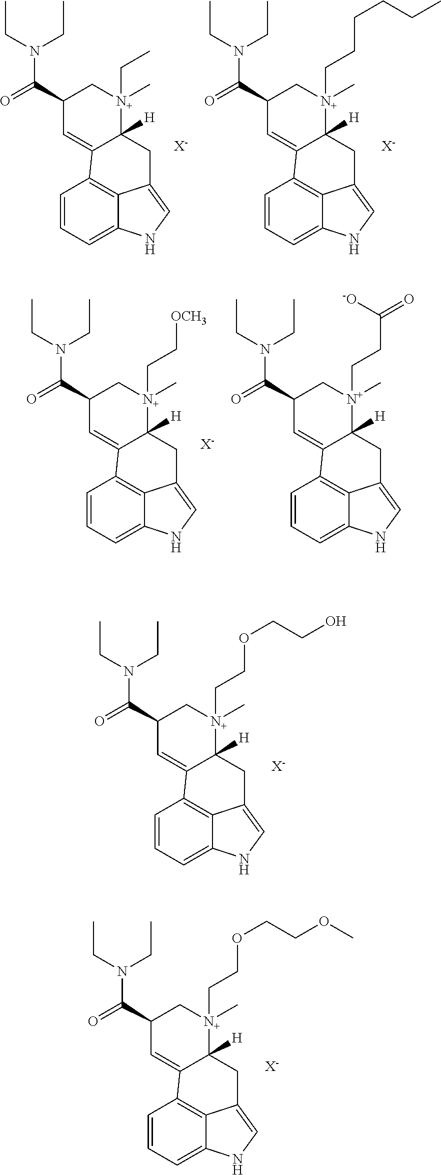

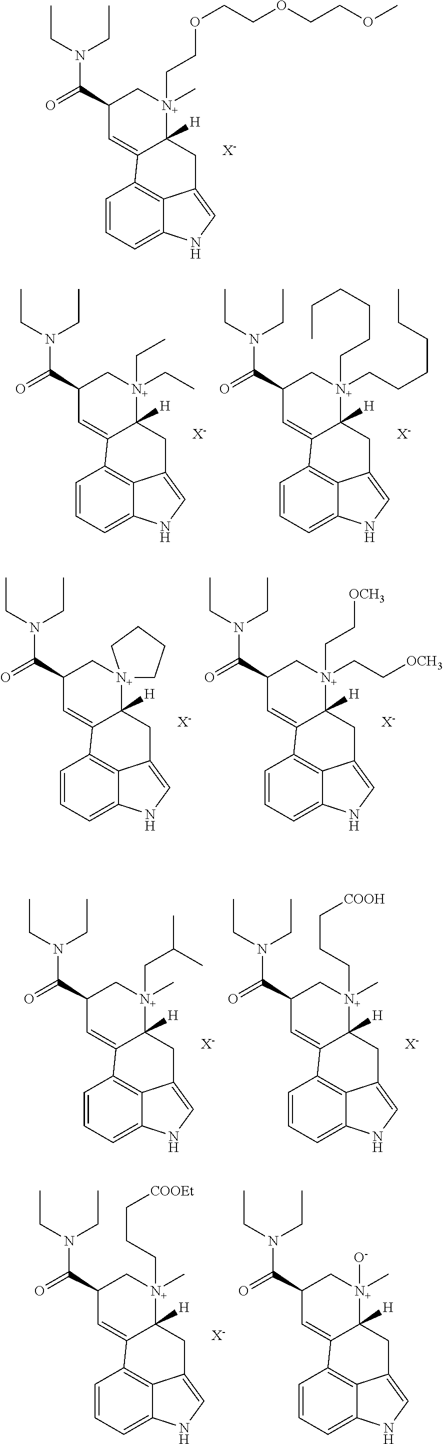

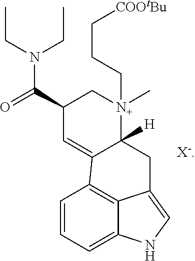

15. (canceled)

16. A pharmaceutical composition comprising a compound represented by Formula (I), 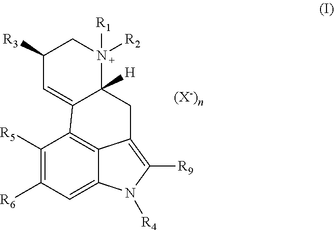
wherein
R₁ and R₂ are independently selected from the group consisting of substituted or unsubstituted C₁-C₁₂-alkyl, C₂-C₁₂-alkenyl; —CH₂CH₂—(OCH₂CH₂)ₘOR₁₂; and C₁-C₁₂-alkyl-Y; or
one of R₁ and R₂ is hydroxyl; or
R₁ and R₂ are taken together with nitrogen atom to which they are attached to form a substituted or unsubstituted 3- to 8-membered heterocyclyl;
R₃ is selected from the group consisting of hydrogen, halogen, substituted or unsubstituted C₁-C₆-alkyl, —OR₈, —NR₇R₈, —C(O)NR₇R₈, —C(O)O⁻, and —C(O)OR₈;
R₄ is selected from the group consisting of substituted or unsubstituted C₁-C₆-alkyl, C₁-C₆-alkyl-OR₁₀, C₁-C₆-alkyl-NR₁₀R₁₁, —C(O)R₁₀, and —C(O)NR₁₀R₁₁;
R₅ and R₆ are independently selected from the group consisting of hydrogen, halogen, hydroxyl, optionally substituted C₁-C₆-alkyl and C₁-C₆-alkoxy;
R₇ and R₈ are independently selected from the group consisting of hydrogen and substituted or unsubstituted C₁-C₆-alkyl; or R₇ and R₈ are taken together with nitrogen atom to which they are attached to form a substituted or unsubstituted 3- to 8-membered heterocyclyl;
R₉ is selected from the group consisting of hydrogen, halogen, and substituted or unsubstituted C₁-C₆-alkyl;
R₁₀ and R₁₁ are independently selected from the group consisting of hydrogen and C₁-C₆-alkyl;
R₁₂ is hydrogen or C₁-C₆-alkyl;
Y is selected from the group consisting of hydroxyl, substituted or unsubstituted C₁-C₆-alkoxy; substituted or unsubstituted C₁-C₆-alkyl-OC(O)—; and —C(O)O⁻;
X⁻ is a pharmaceutically acceptable anion;
m is an integer from 1 to 5;
n is 0 when at least one of R₃ or Y is —C(O)O⁻; and
n is 1 when neither R₃ nor Y is —C(O)O⁻;
and a pharmaceutically acceptable carrier or excipient.

17. The pharmaceutical composition of claim 16 wherein the compound is represented by Formula (II),

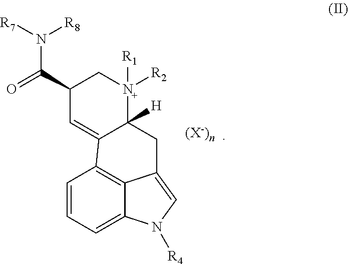

18. The pharmaceutical composition of claim 17 wherein R₇ and R₈ are independently C₁-C₆-alkyl.

19. (canceled)

20. The pharmaceutical composition of claim 16 wherein R₁ and R₂ are independently C₁-C₁₂-alkyl or C₁-C₁₂-alkyl-Y.

21-28. (canceled)

29. The pharmaceutical composition of claim 16, wherein the compound is selected from the compounds set forth below:

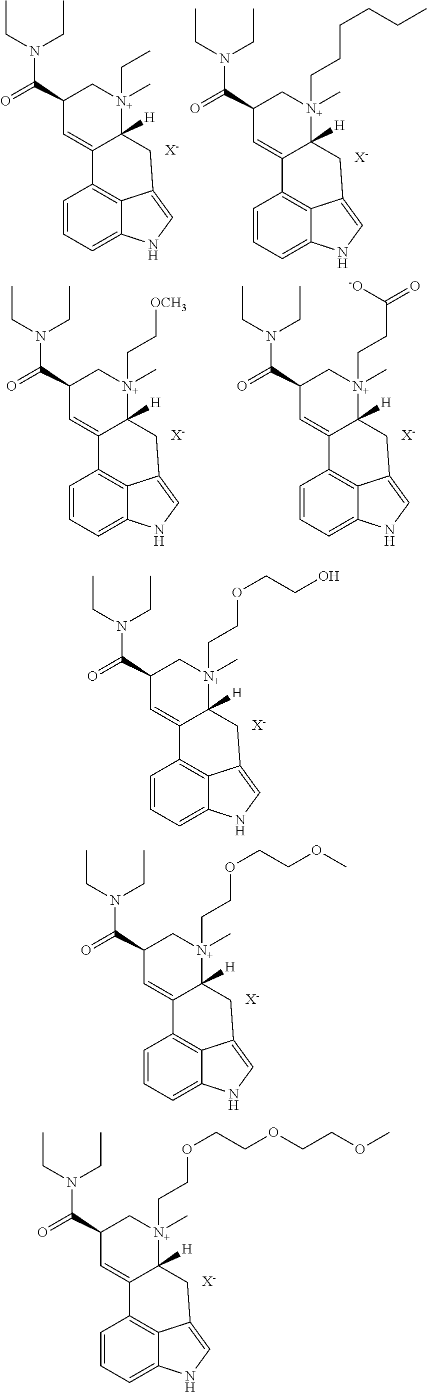

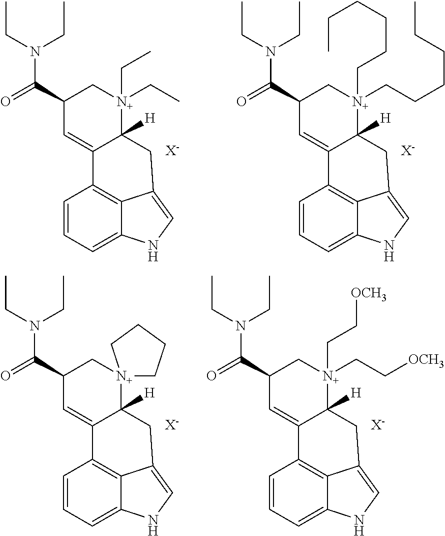

30. (canceled)

31. A method for treating neuropathic pain or pruritis in a subject in need thereof, comprising administering to the subject a therapeutically effective amount of a compound represented by Formula (I), 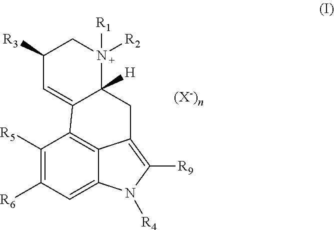
wherein
R₁ and R₂ are independently selected from the group consisting of substituted or unsubstituted C₁-C₁₂-alkyl, C₂-C₁₂-alkenyl; —CH₂CH₂—(OCH₂CH₂)ₘOR₁₂; and C₁-C₁₂-alkyl-Y; or
R₁ and R₂ are taken together with nitrogen atom to which they are attached to form a substituted or unsubstituted 3- to 8-membered heterocyclyl;
R₃ is selected from the group consisting of hydrogen, halogen, substituted or unsubstituted C₁-C₆-alkyl, —OR₈, —NR₇R₈, —C(O)NR₇R₈, —C(O)O, and —C(O)OR₈;
R₄ is selected from the group consisting of substituted or unsubstituted C₁-C₆-alkyl, C₁-C₆-alkyl-OR₁₀, C₁-C₆-alkyl-NR₁₀R₁₁, —C(O)R₁₀, and —C(O)NR₁₀R₁₁;
R₅ and R₆ are independently selected from the group consisting of hydrogen, halogen, hydroxyl, optionally substituted C₁-C₆-alkyl and C₁-C₆-alkoxy;
R₇ and R₈ are independently selected from the group consisting of hydrogen and substituted or unsubstituted C₁-C₆-alkyl; or R₇ and R₈ are taken together with nitrogen atom to which they are attached to form a substituted or unsubstituted 3- to 8-membered heterocyclyl;
R₉ is selected from the group consisting of hydrogen, halogen, and substituted or unsubstituted C₁-C₆-alkyl;
R₁₀ and R₁₁ are independently selected from the group consisting of hydrogen and C₁-C₆-alkyl;
R₁₂ is hydrogen or C₁-C₆-alkyl;
Y is selected from the group consisting of hydroxyl, substituted or unsubstituted C₁-C₆-alkoxy; substituted or unsubstituted C₁-C₆-alkyl-OC(O)—; and —C(O)O⁻;
X⁻ is a pharmaceutically acceptable anion;
m is an integer from 1 to 5;
n is 0 when at least one of R₃ or Y is —C(O)O⁻; and
n is 1 when neither R₃ nor Y is —C(O)O⁻;
and a pharmaceutically acceptable carrier or excipient.

32. The method of claim 31 wherein the compound is represented by Formula (II),

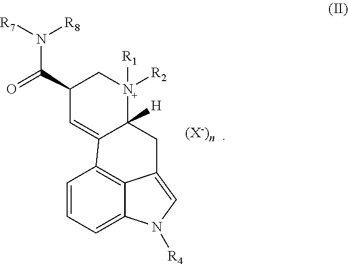

33. The method of claim 32 wherein R₇ and R₈ are independently C₁-C₆-alkyl.

34. (canceled)

35. The method of claim 31 wherein R₁ and R₂ are independently C₁-C₁₂-alkyl or C₁-C₁₂-alkyl-Y.

36-43. (canceled)

44. The method of claim 31 wherein the compound is selected from the compounds set forth below:

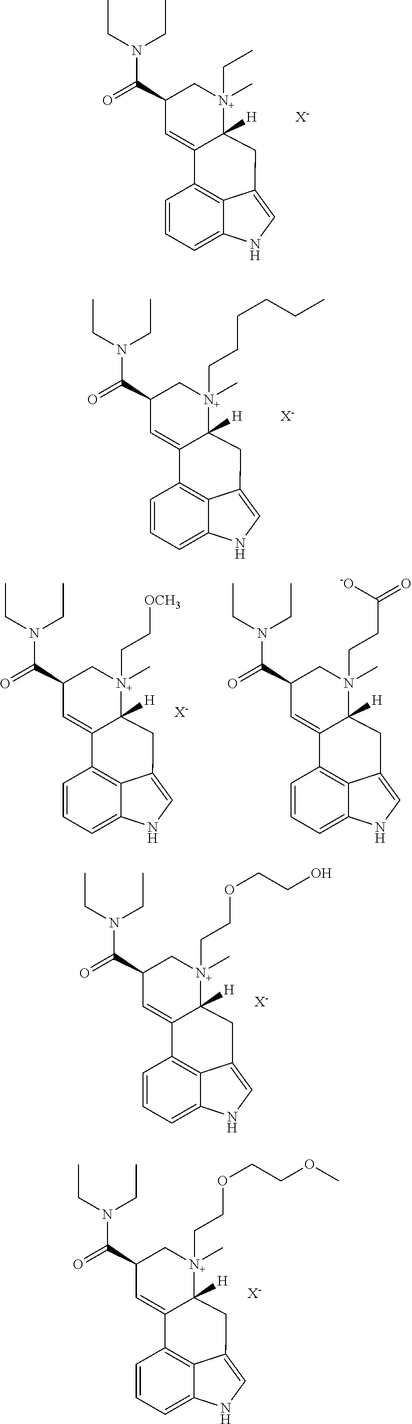

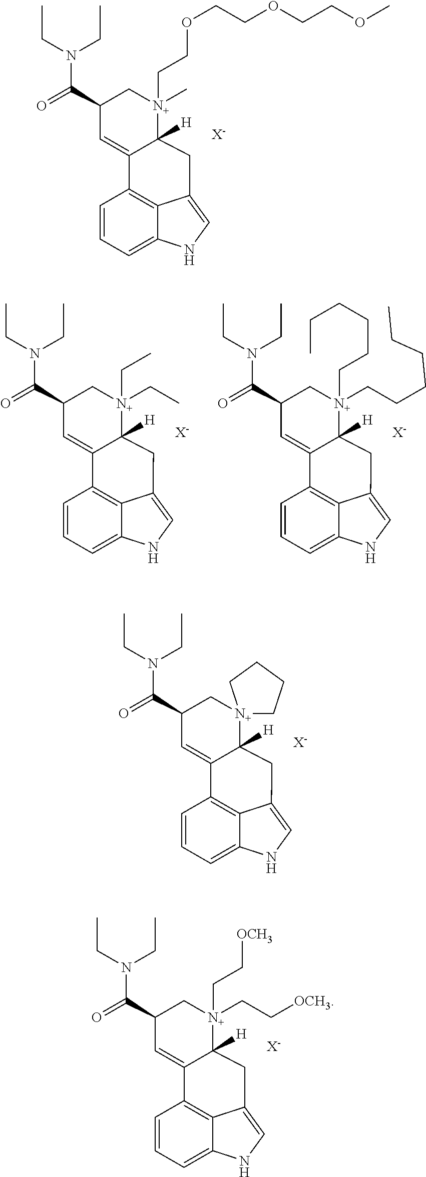

45. (canceled)

46. The method of claim 31 wherein the method is for treating neuropathic pain.

47-49. (canceled)

50. The method of claim 31 wherein the method is for treating pruritis.

51. (canceled)

52. (canceled)

53. The method of claim 31 wherein the method is for treating nociceptive pain or nociplastic pain.

54-59. (canceled)
